(19) United States
(12) Patent Application Publication
(10) Pub. No.: US 2004/0142877 A1
(43) Pub. Date: Jul. 22, 2004

(54) Ligands of the $g(a)v$g(b)6 integrin

(76) Inventors: Oliver Schadt, Rodenbach (DE); Alfred Jonczyk, Darmstadt (DE); Wolfgang Staehle, Ingelheim (DE); Simon Goodman, Griesheim (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/474,802

(22) PCT Filed: Mar. 13, 2002
(86) PCT No.: PCT/EP02/02728

(30) Foreign Application Priority Data
Apr. 14, 2001 (DE) 101185502

(51) Int. Cl. A61K 38/04; C07K 5/04

(52) U.S. Cl. 514/19; 562/450; 546/335; 548/204; 548/236; 548/338.1; 558/414

(57) ABSTRACT

Novel biphenyl derivatives of the general formula I

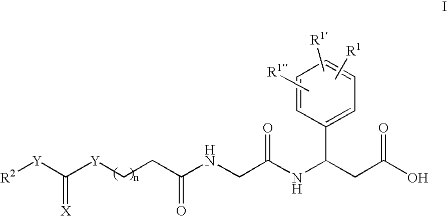

in which R¹, R^{1′}, R^{1″}, R² and n are as defined in claim 1, their stereoisomers and their physiologically acceptable salts or solvates are novel integrin ligands, in particular of the αᵥβ₆ integrin receptor. The novel compounds can be used as medicaments.

The invention relates to novel compounds of the formula I

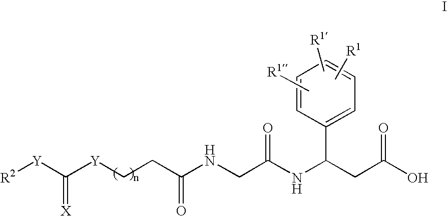

in which

X is O or S

Y independently of one another are NH, O or S

R¹, R^{1′} and R^{1″} are H, A, Ar, Het, Hal, NO₂, CN, OH, OA, NH₂, NHA, NA₂, COOH, COOA, CONH₂, CONHA or CONA₂

R² is H, A, alkenyl having from 1 to 8 carbon atoms and from 1 to 2 double bonds, (CH₂)ₘAr, (CH₂)ₘHet, (CH₂)ₘcycloalkyl, (CH₂)ₘCHAAr, (CH₂)ₘCHAHet or (CH₂)ₘCHA-cycloalkyl,

A is alkyl having from 1 to 8 carbon atoms,

Het is an aromatic monocyclic or bicyclic heterocyclic radical having from 1 to 4 N, O and/or S atoms, which may be unsubstituted or monosubstituted or disubstituted by Hal, A, OH, OA, SA, OCF₃, —CO—A, CN, COOA, COOH, CONH₂, CONHA, CONA₂, NH₂, NHA, NA₂ and/or NO₂,

m is 0, 1 or 2

n is 1,2, 3 or 4,

their stereoisomers and their physiologically acceptable salts and solvates.

Compounds having a partially similar structure are disclosed in WO 96/22966 A1, WO 97/08145 A1 and WO 00/48996 A2, where all compounds are effective as integrin inhibitors. Integrins are membrane-bound, heterodimeric glycoproteins which consist of an α-subunit and a smaller β-subunit. The relative affinity and specificity for ligand binding is determined by the combination of the different α- and β-subunits. According to the disclosure content of the said patent applications, the compounds of WO 96/22966 A1 selectively inhibit the α₄β₁ integrin receptor, and the compounds of WO 97/08145 A1 selectively inhibit the αᵥβ₃ integrin receptor. The compounds of WO 00/48996 A2 inhibit principally αᵥβ₃ and αᵥβ₅ integrin receptors.

The invention had the object of finding novel compounds having valuable properties, in particular those which are used for the preparation of medicaments.

It has been found that the compounds of the formula I and their salts have very valuable pharmacological properties and are well tolerated. Surprisingly, the novel compounds according to the invention are preferred ligands of the αᵥβ₆ integrin receptor.

The compounds act, in particular, as antagonists, but can also act as agonists. Whereas agonists have both affinity and intrinsic activity and stimulate receptors, antagonists inhibit the stimulating action of agonists.

The integrins are ascribed various physiological and pathological functions, which are revealed in detail, for example, by the following review papers: Integrins and signal transduction. Dedhar-S, Curr-Opin-Hematol. 1999 January; 6(1): 37-43, Integrins take partners: cross-talk between integrins and other membrane receptors. Porter-J C; Hogg-N, Trends-Cell-Bio. 1998 October; 8(10): 390-6, Regulation of integrin-mediated adhesion during cell migration. Cox-E A; Huttenlocher-A, Microsc-Res-Tech. 1998 Dec. 1; 43(5): 412-9, The role of integrins in the malignant phenotype of gliomas. Uhm-J H; Gladson-C L; Rao-J S, Front-Biosci. 1999 Feb. 15; 4: D188-99, or Sperm disintegrins, egg integrins, and other cell adhesion molecules of mammalian gamete plasma membrane interactions. Evans-J P Front-Biosci. 1999 Jan. 15; 4: D114-31.

An important role here is ascribed to the αᵥ integrins, as found, for example, in The role of alpha v-integrins in tumour progression and metastasis. Marshall-J F; Hart-I R Semin-Cancer-Biol. 1996 June; 7(3): 129-38 or The role of alpha v-integrins during angiogenesis. Eliceiri-B P and Cheresh-D A Molecular Medicine 4: 741-750 (1998).

These integrins also include αᵥβ₆ Epithelial integrins. Sheppard-D Bioessays. 1996 August; 18(8): 655-60 and the two integrins αᵥβ₃ and αᵥβ₅, which are known adhesion receptors, whose biological importance has been referred to, for example, in J. A. Varner et al. Cell Adhesion and Communication 3, 367-374 (1995) and in J. Samanen et al. Curr. Pharmaceutical Design, 3, 545-584 (1997).

αᵥβ₆ is a relatively rare integrin (Busk et al., 1992 J. Biol. Chem. 267(9), 5790), which is increasingly formed in epithelial tissue during repair processes and preferentially binds the natural matrix molecules fibronectin and tenascin (Wang et al., 1996, Am. J. Respir. Cell Mol. Biol. 15(5), 664). In addition, vitronectin also binds to αᵥβ₆ (Characterization of the integrin alpha v beta 6 as a fibronectin-binding protein. Busk-M; Pytela-R; Sheppard-D. J-Biol-Chem. 1992 Mar. 25; 267(9): 5790-6; Restricted distribution of integrin beta 6 mRNA in primate epithelial tissues. Breuss,-J-M; Gillett, -N; Lu,-L; Sheppard,-D; Pytela, -R J-Histochem-Cytochem. 1993 October; 41(10): 1521-7; Differential regulation of airway epithelial integrins by growth factors. Wang-A; Yokosaki-Y; Ferrando-R; Balmes-J; Sheppard-D. Am-J-Respir-Cell-Mol-Biol. 1996 Nov.; 15(5): 664-72); The integrin alphavbeta6 is critical for keratinocyte migration on both its known ligand, fibronectin, and on vitronectin. Huang, -X; Wu,-J; Spong, -S; Sheppard,-D J-Cell-Sci. 1998 August; 111 (Pt 15)2189-95).

The physiological and pathological functions of αᵥβ₆ are still not known precisely, but it is assumed that this integrin plays an important role in physiological processes and disorders (for example inflammation, wound healing and tumours) in which epithelial cells are involved (Expression of the beta 6 integrin subunit in development, neoplasia and tissue repair suggests a role in epithelial remodeling. Breuss,-J-M; Gallo,-J; DeLisser, -H-M; Klimanskaya,-I-V; Folkesson, -H-G; Pittet,-J-F; Nishimura, -S-L; Aldape,-K; Landers,-D-V; Carpenter, -W; et-al. J-Cell-Sci. 1995 June; 108 (Pt 6)2241-51).

Thus, αᵥβ₆ is expressed on keratinocytes in wounds (Keratinocytes in human wounds express alpha v beta 6 integrin. Haapasalmi-K, Zhang-K, Tonnesen-M, Olerud-J, Sheppard-D, Sblo-T, Kramer-R, Clark-RA, Uitto-VJ, Larjava-H. J-Invest-Dermatol. 1996 January, 106(1): 42-8; Epidermal integrin expression is upregulated rapidly in human fetal wound repair. Cass-D-L, Bullard-K-M, Sylvester-K-G, Yang-E-Y, Sheppard-D, Herlyn-M, Adzick-N-S J-Pediatr-Surg. 1998 February, 33(2): 312-6), from which it can be assumed that, besides wound-healing processes and inflammation, other pathological occurrences in the skin, such as, for example, psoriasis, can be influenced by agonists or antagonists of the said integrin.

Furthermore, in disturbed hornification of the skin (in the mucosa of the oral cavity, at the lips, the tongue and the genitals), so-called leukoplakia, αᵥβ₆ is expressed to a greater extent compared with normal comparative tissue. The frequency and level of expression of the leukoplakia increases, via lichen planus, to squamous cell carcinoma, and consequently a correlation between expression of αᵥβ₆ and the malign transformation of leukoplakia is assumed: Expression of alpha(v)beta6 integrin in oral leukoplakia. Hamidi-S, Salo-T, Kainulainen-T, Epstein-J, Lemer-K, Ladava-H Br-J-Cancer. 2000 April, 82(8): 1433-40; Stromal fibroblasts influence oral squamous-cell carcinoma cell interactions with tenascin-C. Ramos-D-M, Chen-B-L, Boylen-K, Stem-M, Kramer-R-H, heppard-D, Nishimura-S-L, Greenspan-D, Zardi-L, Pytela-R lnt-J-Cancer. 1997 Jul. 17, 72(2): 369-76; Expression of the alpha v beta 6 integrin promotes migration and invasion in squamous carcinoma cells Thomas-G J, Lewis-M P, Whawell-S A, Russell-A, Sheppard-D, Hart-I R, Speight-P M, Marshall-J F JOURNAL-OF-INVESTIGATIVE-DERMATOLOGY. JULY 2001; 117 (1): 67-73; Integrins alpha5beta1, alphavbeta 1, and alphavbeta6 collaborate in squamous carcinoma cell spreading and migration on fibronectin. Koivisto,-L, Grenman-R, Heino-J, Larjava-H Exp-Cell-Res. 2000 Feb 25, 255(1): 10-7).

Furthermore, αᵥβ₆ plays a role in the respiratory tract epithelium (Weinacker et al., 1995, Am. J. Respir. Cell Mol. Biol. 12(5), 547-56; Expression of the human integrin beta6 subunit in alveolar type II cells and bronchiolar epithelial cells reverses lung inflammation in beta6 knockout mice. Huang X, Wu J, Zhu W. Pytela R, Sheppard D, Am-J-Respir-Cell-Mol-Biol. 1998 Oct, 19(4): 636-42; Expression of integrin cell adhesion receptors during human airway epithelial repair in vivo. Pilewski J M, Latoche J D, Arcasoy S M, Albelda-S-M Am-J-Physiol. 1997 July, 273(1 Pt 1): L256-63; Global analysis of gene expression in pulmonary fibrosis reveals distinct programs regulating lung inflammation and fibrosis. Kaminski, -N; Allard J D, Pittet J F, Zuo F, Griffiths M J, Morris D, Huang X, Sheppard D, Heller R A, Proc-Nati-Acad-Sci-U-S-A . 2000 Feb 15, 97(4): 1778-83), and consequently corresponding agonists/antagonists of this integrin could successfully be employed in respiratory tract disorders, such as bronchitis, asthma, lung fibrosis and respiratory tract tumours.

Besides the lung (bronchi), fibrosis may also occur in other organs, such as, for example, in the skin, the liver (extending to cirrhosis), the kidney and the bladder, the heart and the pancreas (cystic fibrosis). It is assumed that the integrin αᵥβ₆ also plays a role in this pathological connective tissue proliferation, and the course of the illness can therefore be influenced by agonists/antagonists of integrin αᵥβ₆ (Mechanisms of tissue repair from wound healing to fibrosis, Mutsaers S E, Bishop J E, Mcgrouther G, Laurent G, J Int. J. Biochem. Cell Biol. (1997) 29(1): 5-17; avb6 Integrin mediates latent TGFβ activation: Implications for cutaneous fibrosis. Dalton S L, J. Am. Acad. Dermatol (1999) 41: 457-463; Clinical significance of blood serum connective tissue components in organ fibrosis, Kropf J, Gressner AM, Z. Med. Laboratoriumsdiagn. (1991) 32(3/4): 150-8, Angiotensin II, adhesion, and cardiac fibrosis, Schnee J M, Hsueh W A, Cardiovasc. Res. (2000) 46(2): 264-268; Pulmonary fibrosis and its treatment: today and in the next millennium. Sime P, J. Curr. Opin. Anti-Inflammatory Immunomodulatory Invest. Drugs (1999) 1(5): 423-432; Hepatic fibrosis: pathophysiology and laboratory diagnosis, Housset C, Guechot J, Pathol. Biol. (1999) 47(9): 886-894; Progressive renal disease. Fibroblasts, extracellular matrix, and integrins, Norman J T, Fine L G, Exp. Nephrol. (1999) 7(2): 167-177, Renal fibrosis: insights into pathogenesis and treatment, Nahas A M E I, Muchaneta-Kubara E C, Essawy M, Soylemezoglu O, Int. J. Biochem. Cell Biol. (1997) 29(1): 55-62).

It is furthermore known that αᵥβ₆ also plays a role in the intestinal epithelium, and consequently corresponding integrin agonists/antagonists could be used in the treatment of inflammation, tumours and wounds of the stomach/intestinal tract. There are indications here that integrin αᵥβ₆ also influences the secretion of matrix metalloproteases, such as, for example, that of gelatinase B (MMP-9): The alpha v beta 6 integrin promotes proliferation of colon carcinoma cells through a unique region of the beta 6 cytoplasmic domain, Agrez M, Chen A, Cone R I, Pytela R, Sheppard D, J Cell Biol (1994) 127(2): 547-56; Integrin-mediated signalling of gelatinase B secretion in colon cancer cells, Niu J, Gu X, Turton J, Meldrum C, Howard E W, Agrez M, Biochem Biophys Res Commun (1998) 249(1):287-91.

It has been found that the expression of αᵥβ₆ is accompanied by changes in the cell density and MMP activity (The alpha v beta 6 integrin regulates its own expression with cell crowding: Implications for tumour progression, Niu J, G u X, Ahmed N, Andrews S, Turton J, Bates R, Agrez M, INTERNATIONAL JOURNAL OF CANCER, (2001) 92 (1):40-48; The alpha v beta 6 integrin induces gelatinase B secretion in colon cancer cells, Agrez M, Gu X, Turton J, Meldrum C, Niu J, Antalis T, Howard E W, Int J Cancer (1999) 81(1): 90-7; alpha v beta 6 integrin upregulates matrix metalloproteinase 9 and promotes migration of normal oral keratinocytes, Thomas G J, Poomsawat S, Lewis M P, Hart I R, Speight P M, Marshall J F, JOURNAL OF INVESTIGATIVE DERMATOLOGY (2001) 116 (6): 898-904; alpha V beta 6 integrin promotes invasion of squamous carcinoma cells through up-regulation of matrix metalloproteinase-9, Thomas G J, Lewis M P, Hart I R, Marshall J F, Speight P M, INTERNATIONAL JOURNAL OF CANCER (2001) 92 (5): 641-650). Regulation of the MMP activity (possibility different MMPs) by tumour cells as a function of their density could thus be a mechanism which enables the cells to re-create space for proliferation and migration by proteolysis of the surrounding matrix during growth of the tumour mass.

Owing to the role of integrin αᵥβ₆ in infection processes, it is assumed that its agonists/antagonists can also be used in microbial infections (protozoa, microphytes, bacteria, viruses, yeasts and fungi). The correlation with integrin αᵥβ₆ has been described, for example, for the coxsackievirus or for infection of host cells with the foot-and-mouth disease virus (FMDV), which proceeds αᵥβ₃-dependently, but can also take place αᵥβ₆-dependently (Integrin alpha v beta 6 enhances coxsackievirus B1 lytic infection of human colon cancer cells. Agrez M V, Shafren D R, G u X, Cox K, Sheppard D, Barry R D, Virology (1997) 239(1): 71-7; The epithelial integrin alphavbeta6 is a receptor for foot-and-mouth disease virus, Jackson T, Sheppard D, Denyer M, Blakemore W, King A M, J Virol (2000) 11: 4949-56; Role of the cytoplasmic domain of the beta-subunit of integrin alpha(v)beta6 in infection by foot-and-mouth disease virus, Miller L C, Blakemore W. Sheppard D, Atakilit A, King A M, Jackson T, J Virol (2001) 75(9): 4158-64; The ability of integrin avb3 to function as a receptor for foot-and-mouth disease virus is not dependent on the presence of complete subunit cytoplasmic domains, Neff S, Baxt B, J Virol (2001) 75(1): 527-532, Foot-and-mouth disease virus virulent for cattle utilizes the integrin avb3 as its receptor, Neff S, Sa-Carvalho D, Rieder E, Mason, P W, Blystone S D, Brown E J, Baxt B, J Virol (1998) 72(5): 3587-3594; Arginine-glycine-aspartic acid-specific binding by foot-and-mouth disease viruses to the purified integrin avb3 in vitro, Jackson T, Sharrna A, Ghazaleh. R A, Blakemore W E, Ellard F M, Simmons D L, Newman J W I, Stuart D I, King A M Q, J Virol (1997) 71(II): 8357-8361).

Infection with HIV (AIDS) is also dependent on αᵥβ integrins, and consequently the agonists/antagonists of integrin αᵥβ₆ would likewise be employed here (A novel integrin specificity for the human immunodeficiency virus (HIV) Tat protein, Ruoslahti E I, Vogel B E, Wong-Staal F Y, PCT Int. Appl (1992) WO 9214755).

According to more recent knowledge, the bacterium Bacillus anthracis secretes a toxin which consists of 3 proteins, one of which, the so-called PA or protective antigen, binds to receptors on the cell membrane (anthrax toxin receptor, ATR). ATR is a type I membrane protein with an extracellular domain of the Willebrandt factor type (vWF A). Integrins also contain vWF A domains of this type. This is comprehensible via a homology analysis in the Swiss Prot database both for integrin αᵥβ₆ (http://www.expasy.ch/cgi-bin/niceprot. pl?P18564; sequence β₆ (131-371)) here, and also for αᵥβ₃ (http://www.expasy.ch/cgi-bin/niceprot.pl?PO5106; β₃ (135-377)). It is therefore assumed that αᵥβ₆ agonists/antagonists can also be used for anthrax of the lung, skin and intestine) (Identification of the cellular receptor for anthrax toxin. K. A. Bradley et al. Nature 414, 225-229 (2001) [and accompanying articles]; Evolution of von Willebrand factor A (vWA) domains, Tuckwell D, Biochem Soc Trans (1999) 27(6): 835-840).

The dependence of the infection of host cells on their adhesion receptors for bacteria and for yeasts (budding fungi, candida) (Cell adhesion molecules in the pathogenesis of and host defence against microbial infection, Kerr J R, Medical Microbiology, Manchester Royal Infinnary, UK, MOLECULAR PATHOLOGY (1999) 52(4): 220-30; Vitronectin-dependent invasion of epithelial cells by Neisseria gonorrhoeae involves alpha(v) integrin receptors, Dehio M, Gomez-Duarte O G, Dehio C, Meyer T F, FEBS LETTERS (1998) 424(1-2): 84-8; A natural variant of the cysteine protease virulence factor of group A Streptococcus with an arginine-glycine-aspartic acid (RGD) motif preferentially binds human integrins alphavbeta3 and alphaIIbbeta3, Stockbauer K E, Magoun L, Liu M, Bums E H Jr, Gubba S, Renish S, Pan X, Bodary S C, Baker E, Coburn J, Leong J M, Musser J M, PROCEEDINGS OF THE NATIONAL ACADEMY OF SCIENCES OF THE UNITED STATES OF AMERICA (1999), 96(1): 242-7; Involvement of alpha(v)beta3 integrin-like receptor and glycosaminoglycans in Candida albicans germ tube adhesion to vitronectin and to a human endothelial cell line, Santoni G, Spreghini E, Lucciadni R, Amantini C, Piccoli M, MICROBIAL PATHOGENESIS (2001) 31(4): 159-72) indicates the possibility of using agonists/antagonists of integrin αᵥβ₆ in these cases too.

Integrin αᵥβ₆ interacts with TGF-β, resulting in its activation (avb6 Integrin mediates latent TGFβ activation: Implications for cutaneous fibrosis, Dalton S L, J Am Acad Dermatol (1999) 41: 457-463; The integrin avb6 binds and activates latent TGFb1: a mechanism for regulating pulmonary inflammation and fibrosis, Munger J S et al. Cell (1999) 96: 319-328). Latent TGFβ₁ (one of the pro-forms) binds to integrin αᵥβ₆ and is proteolytically activated thereby. The integrin αᵥβ₆ agonists/antagonists according to the invention could thus prevent activation of TGF-β₁ and other sub-types by inhibiting the binding of TGF-β (pro-form, LAP peptide, LAP-TGFβ, latent TGF) and thereby modulate the effect of TGFβ.

3 human TGFβ isoforms have been discovered to date, which are ascribed a role in a multiplicity of growth and differentiation processes, but in particular in inflammatory processes, fibrosis, wound healing, bone growth, the modulation of immunofunctions, in angiogenesis and tumour metastasis (Rifkin DB et al., Thrombosis and Haemostasis (1993) 70: 177-179; Hata A et al., Molecular Medicine Today (June 1998) 257-262; Integrin-mediated activation of transforming growth factor-beta(1) in pulmonary fibrosis, Sheppard DC, (2001) 120(1 Suppl): 49S-53S; Wickstom P et al., Prostate (1998) 37: 19-29). The αᵥβ₆ agonists/antagonists according to the invention could thus also be employed in these processes.

A further paper which emphasises the role of αᵥβ₆ in immunological processes describes the influx of neutrophiles after chemical damage to the lung (Expression of the beta6 integrin subunit is associated with sites of neutrophil influx in lung epithelium, Miller LA, Bamett NL, Sheppard D, Hyde DM, J Histochem Cytochem (2001) 49(1): 41-8).

The effect of a compound on an αᵥβ₆ integrin receptor and thus on the activity as an inhibitor can be demonstrated, for example, by the method described by J. W. Smith et al. in J. Biol. Chem. 1990, 265, 12267-12271.

Besides the preferred inhibition of αᵥβ₆ integrin receptors, the compounds also act as inhibitors of αᵥβ₃ or αᵥβ₅ integrin receptors and as inhibitors of glycoprotein IIb/IIIa. Integrin αᵥβ₃, for example, is expressed on a number of cells, for example endothelial cells, cells of the smooth vascular muscles, for example of the aorta, cells for the breakdown of bone matrix (osteoclasts) or tumour cells.

The action of the -compounds according to the invention can be demonstrated, for example, by the method described by J. W. Smith et al. in J. Biol. Chem. 1990, 265,12267-12271.

The dependence of formation of angiogenesis on the interaction between vascular integrins and extracellular matrix proteins has been described by P. C. Brooks, R. A. Clark and D. A. Cheresh in Science 1994, 264, 569-571.

The possibility of inhibiting this interaction and so initiating apoptosis (programmed cell death) of angiogenic vascular cells by a cyclic peptide has been described by P. C. Brooks, A. M. Montgomery, M. Rosenfeld, R. A. Reisfeld, T. H u, G. Klier and D. A. Cheresh in Cell 1994, 79,1157-1164. In this, for example, αvβ3 antagonists or antibodies against αvβ3 were described which cause shrinkage of tumours due to the initiation of apoptosis.

The experimental evidence that the compounds according to the invention also prevent the attachment of living cells to the corresponding matrix proteins and accordingly also prevent the attachment of tumour cells to matrix proteins can be provided in a cell adhesion test analogously to the method of F. Mitjans et al., J. Cell Science 1995,108,2825-2838.

The compounds of the formula I are able to inhibit the binding of metallo-proteinases to integrins and thus prevent the cells utilising the enzymatic activity of the proteinase. An example can be found in the ability of a cyclo-RGD peptide to inhibit the binding of MMP-2 (matrix-metallo-proteinase-2) to the vitronectin receptor αvβ3, as described in P. C. Brooks et al., Cell 1996, 85, 683-693.

Compounds of the formula I which block the interaction of integrin receptors and ligands, such as, for example, of fibrinogen to the fibrinogen receptor (glycoprotein (Ib/IIIa), prevent, as antagonists, the spread of tumour cells by metastasis and can therefore be employed as antimetastatic substances in operations in which tumours are removed or attacked surgically. This is confirmed by the following observations:

The spread of tumour cells from a local tumour into the vascular system occurs through the formation of microaggregates (microthromboses) due to the interaction of the tumour cells with blood platelets. The tumour cells are masked by the protection in the microaggregate and are not recognised by the immune system cells. The microaggregates are able to attach to vessel walls, simplifying further penetration of tumour cells into the tissue. Since the formation of microthromboses is promoted by ligand binding to the corresponding integrin receptors, for example αvβ3 or allbβ3, on activated blood platelets, the corresponding antagonists can be regarded as effecttive metastasis inhibitors.

The action of a compound on an αvβ5 integrin receptor and thus the activity as an inhibitor can be demonstrated, for example, by the method described by J. W. Smith et al. in J. Biol. Chem. 1990, 265, 12267-12271.

A measure of the uptake of a medicament active ingredient in an organism is its bioavailability.

If the medicament active ingredient is administered to the organism intravenously in the form of an injection solution, its absolute bioavailability, i.e. the proportion of the pharmaceutical species which is unchanged in the systemic blood, i.e. enters the general circulation, is 100%.

On oral administration of a therapeutic active ingredient, the active ingredient is generally in the form of a solid in the formulation and must therefore first dissolve in order that it can overcome the entry barriers, for example the gastrointestinal tract, the oral mucous membrane, nasal membranes or the skin, in particular the stratum corneum, and can be absorbed by the body. Pharmacokinetic data, i.e. on the bioavailability, can be obtained analogously to the method of J. Shaffer et al., J. Pharm. Sciences, 1999, 88, 313-318.

A further measure of the absorbability of a therapeutic active ingredient is the logD value, since this value is a measure of the lipophilicity of a molecule.

The compounds of the formula I have at least one centre of chirality and can therefore occur in a number of stereoisomeric forms. All of these forms (for example D and L forms) and mixtures thereof (for example the DL forms) are included in the formula.

The compounds according to the invention according to claim 1 also include so-called prodrug derivatives, i.e. compounds of the formula I modified with, for example, alkyl or acyl groups, sugars or oligopeptides, which are rapidly cleaved in the organism to give the effective compounds according to the invention.

Furthermore, free amino groups or free hydroxyl groups can be provided as substituents of compounds of the formula I with corresponding protecting groups.

The term solvates of the compounds of the formula I is taken to mean adductions of inert solvent molecules onto the compounds of the formula I which form owing to their mutual attractive force. Solvates are, for example, mono- or dihydrates or addition compounds with alcohols, such as, for example, with methanol or ethanol.

The invention relates to the compounds of the formula I and their salts and solyates and to a process for the preparation of compounds of the formula I and their salts and solvates, characterised in that

(a) a compound of the formula II

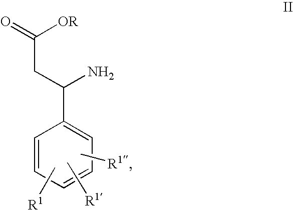

in which R is a protecting group, and R¹, R^{1′} and R^{1″} are as defined in formula I and in which, in the case where R¹, R^{1′} and/or R^{1″} have free hydroxyl or amino groups, these are in each case protected by a protecting group,

is reacted with a compound of the formula III

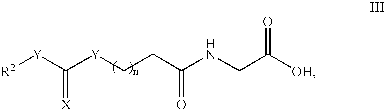

in which R² and n are as defined in formula I and in which, in the case where R² contains free hydroxyl and/or amino groups, these are in each case protected by protecting groups,

and the protecting group R and any protecting groups present on R¹, R^{1″}, R^{1″} and/or R² are removed, or

(b) a compound of the formula IV

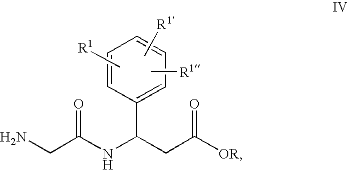

in which R is a protecting group, and R¹, R^{1′} and R^{1″} are as defined in formula I and in which, in the case where R¹, R^{1′} and/or R^{1″} contain free hydroxyl and/or amino groups, these are in each case protected by protecting groups,

is reacted with a compound of the formula V

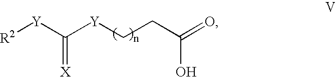

in which n and R² are as defined in formula I and in which, in the case where R² contains free hydroxyl and/or amino groups, these are in each case protected by protecting groups,

and the protecting group R and any protecting groups present on R¹, R^{1′}, R^{1″} and/or R² are removed, or

(c) one or more radicals R¹, R^{1′}, R^{1″} and/or R² in a compound of the formula I are converted into one or more radicals R¹, R^{1′}, R^{1″} and/or R²

by, for example,

i) alkylating a hydroxyl group,

ii) hydrolysing an ester group to a carboxyl group,

iii) esterifying a carboxyl group,

iv) alkylating an amino group,

v) reacting an aryl bromide or iodide with boronic acids by a Suzuki coupling to give the corresponding coupling products, or

vi) acylating an amino group, or

(d) a compound of the formula VI

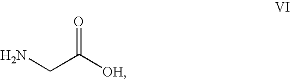

in which the free amino group is protected by protecting groups, is reacted with a compound of the formula II,

in which R is a protecting group and R¹, R^{1′} and R^{1″} are as defined in formula I and in which, in the case where R¹, R^{1′} and/or R^{1″} have free hydroxyl or amino groups, these are in each case protected by a protecting group,

to give a compound of the formula IV,

in which R is a protecting group and R¹, R^{1′} and R^{1″} are as defined in formula I and in which, in the case where R¹, R^{1′} and/or R^{1″} have free hydroxyl or amino groups, these are in each case protected by protecting groups,

the protecting group on the amino group is removed, the compound of the formula IV is subsequently reacted as described in (b) with a compound of the formula V,

in which n and R² are as defined in formula I and in which, in the case where R² contains free hydroxyl and/or amino groups, these are in each case protected by protecting groups,

and the protecting group R and any protecting groups present on R¹, R^{1′}, R^{1″} and/or R² are removed, and/or

a basic or acidic compound of the formula I is converted into one of its salts or solvates by treatment with an acid or base.

Throughout the invention, all radicals which occur more than once, such as, for example, A, may be identical or different, i.e. are independent of one another.

In the above formulae, A is alkyl, is linear or branched, and has from 1 to 8, preferably 1, 2, 3, 4, 5 or 6 carbon atoms. A is preferably methyl, furthermore ethyl, n-propyl, isopropyl, n-butyl, sec-butyl or tert-butyl, furthermore also pentyl, 1-, 2- or 3-methylbutyl, 1,1-, 1,2- or 2,2-dimethylpropyl, 1-ethyl-propyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methyl-propyl, 1,1,2- or 1,2,2-trimethylpropyl, heptyl or octyl. Further preferred embodiments of A are the said alkyl groups, which, however, may be monosubstituted or polysubstituted by Hal or NO₂, preferably trifluoro-methyl, 2,2,2-trifluoroethyl or 2-nitroethyl, or alkyl groups, whose carbon chain may be interrupted by —O—, preferably —CH₂—O—CH₃, —CH₂—O—CH₂—CH₃ or —CH₂—CH₂—O—CH₃.

A is particularly preferably methyl or ethyl.

Alkenyl is linear or branched and has from 2 to 8, preferably 2, 3, 4, 5 or 6 carbon atoms. Alkenyl is preferably ethenyl, 1-propenyl, 2-propenyl, 1-butenyl, 2-butenyl, isobutenyl, 1,4-butadienyl, 1-pentenyl, 2-pentenyl, 2-methyl-1-butenyl, 2-methyl-2-butenyl, 3-methyl-1-butenyl, 1,4-pentadienyl or 1,5-pentadienyl. Particular preference is given to allyl, which has the chemical formula H₂C═CH—CH₂—.

The meanings of the substituents R¹, R¹′ and R¹″ are in each case independent of one another. R¹ is preferably aryl. R¹ is particularly preferably unsubstituted or monosubstituted or disubstituted phenyl, in particular unsubstituted phenyl.

Ar is phenyl, naphthyl, anthranyl, fluorenyl, indenyl, anthracenyl or biphenyl, each of which is unsubstituted or monosubstituted or disubstituted by Hal, A, OA, OH, CO—A, CN, COOA, COOH, CONH₂, CONHA, CONA₂, CF₃, OCF₃ or NO₂. Polysubstituted means disubstituted, trisubstituted or tetrasubstituted, preferably disubstituted or trisubstituted. Prefe-rence is given to phenyl, naphthyl, fluorenyl or biphenyl, each of which is unsubstituted, preferably monosubstituted as indicated. Specific preference is given to naphthyl, phenyl, fluorenyl, o-, m- or p-tolyl, o-, m- or pethylphenyl, o-, m- or p-propylphenyl, o-, m- or p-isopropylphenyl, o-, m- or p-tert-butylphenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-methylthiophenyl, o-, m- or p-methylsulfinylphenyl, o-, m- or p-methylsulfonylphenyl, o-, m- or p-aminophenyl, o-, m- or p-methylaminophenyl, o-, m- or p-dimethylaminophenyl, o-, m- or p-nitrophenyl, o-, m- or p-acetylphenyl, o-, m- or p-methoxycarbonylphenyl, o-, m- or p-aminocarbonylphenyl,

furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro4-methyl-, 2-chloro-5-methyl-, 2-chloro-6-methyl-, 2-methyl-3-chloro-, 2-methyl4-chloro-, 2-methyl-5-chloro-, 2-methyl-6-chloro-, 3-chloro-4-methyl-, 3-chloro-5-methyl- or 3-methyl4-chlorophenyl, 2-bromo-3-methyl-, 2-bromo4-methyl-, 2-bromo-5-methyl-, 2-bromo-6-methyl-, 2-methyl-3-bromo-, 2-methyl4-bromo-, 2-methyl-5-bromo-, 2-methyl-6-bromo-, 3-bromo4-methyl-, 3-bromo-5-methyl- or 3-methyl-4-bromophenyl, 2,4- or 2,5-dinitrophenyl, 2,5- or 3,4-dimethoxyphenyl, 2,3,4-, 2,3,5-, 2,3,6-, 2,4,6- or 3,4,5-trichlorophenyl, 2,4,6-tri-tert-butylphenyl, 2,5-dimethylphenyl, p-iodophenyl, 4-fluoro-3-chlorophenyl, 4-fluoro-3,5-dimethylphenyl, 2-fluoro4-bromophenyl, 2,5-difluoro4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl.

Cycloalkyl has from 3 to 15 carbon atoms and is preferably cyclopropyl, cyclobutyl, cyclopentyl, cyclohexyl, cycloheptyl or cyclooctyl, particularly preferably cyclohexyl. Cycloalkyl is likewise a monocyclic or bicyclic terpene, preferably p-menthane, menthol, pinane, bornane or camphor, where each known stereoisomeric form is included, or adamantyl. For camphor, this is both L-camphor and D-camphor.

Hal is preferably F, Cl or Br. Hal is particularly preferably F or Cl.

Amino-protecting group preferably means formyl, acetyl, propionyl, butyryl, phenylacetyl, benzoyl, tolyl, POA, methoxycarbonyl, ethoxycarbonyl, 2,2,2-trichloroethoxycarbonyl, BOC, 2-iodoethoxycarbonyl, CBZ (“carbo-benzoxy”), 4-methoxybenzyloxycarbonyl, FMOC, Mtr or benzyl.

Het is an unsubstituted, monosubsitituted, disubsitituted, trisubsitituted or tetrasubsitituted heterocyclic radical having 1, 2, 3 and/or 4 N, O and/or S atoms, preferably 1-, 2- or 3-pyrrolyl, 1-, 2-, 4- or 5-imidazolyl, 1-, 3-, 4- or 5-pyrazolyl, 2-, 3- or 4-pyridyl, 2-, 4-, -5- or 6-pyrimidinyl, furthermore preferably 1,2,3-triazol-1-, -4-or-5-yl, 1,2,4-triazol-1-, -3- or 5-yl, 1- or 5-tetrazolyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-benzopyrazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 1H-imidazo[4,5-b]pyridin-2-yl or 1,8-naphthyridin-7-yl, each of which is unsubstituted or mono-substituted or disubstituted by A, NHA and/or NH₂. 4-pyridyl is particularly preferred.

The heterocyclic radicals may also be partially or fully hydrogenated. Het may thus also be, for example, 2,3-dihydro-1-, -2-, -3-, -4- or -5-pyrrolyl, 2,5-dihydro-1-, -2-, -3-, 4- or -5-pyrrolyl, 1-, 2- or 3-pyrrolidinyl, tetrahydro-1-, -2- or 4-imidazolyl, 4,5-dihydroimidazol-2-yl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, morpholinyl, hexahydro-1-, -3- or -4-pyridazinyl, hexahydro-1-, -2-, 4- or -5-pyrimidinyl, 1-, 2- or 3-piperazinyl, 1,2,3,4-tetrahydro-1-, -2-, -3-, 4-, -5-, -6-, -7- or -8-quinolyl, 1,2,3,4-tetrahydro-1-, -2-, -3-, -4-, -5-, -6-, -7- or -8-isoquinolyl or 1,2,3,4-tetrahydro-1,8-naphthyridin-7-yl.

Hydrogenated or partially hydrogenated Het² radicals may additionally be substituted by ═NH or carbonyl oxygen.

n is preferably 1, 2, 3 or 4, and n is very particularly preferably 2 or 3.

“Poly” substituted means mono-, di-, tri- or tetrasubstituted.

Pol denotes a solid phase with no terminal functional group, as explained in greater detail below. The terms solid phase and resin are used synonymously below.

If the compounds of the formula I contain biphenyl, the second phenyl radical is preferably coupled to the first phenyl radical in the 3- or 4-position, particularly preferably to the 4-position of the first phenyl ring.

Accordingly, the invention relates in particular to the compounds of the formula I in which at least one of the said radicals has one of the preferred meanings indicated above. Some preferred groups of compounds may be expressed by the following sub-formulae Ia to Ii, which conform to the formula I and in which the radicals not designated in greater detail have the meaning indicated under the formula I, but in which

| | | | |
|---|---|---|---|
| in | la) | R² | is H, A, alkenyl, (CH₂)ₘAr, (CH₂)ₘHet, |
| | | | (CH₂)ₘcycloalkyl, (CH₂)ₘCHAAr, |
| | | | (CH₂)ₘCHAHet or (CH₂)ₘCHA-cycloalkyl; |
| in | lb) | R² | is H, A, alkenyl, (CH₂)ₘAr, (CH₂)ₘHet, |
| | | | (CH₂)ₘcycloalkyl, (CH₂)ₘCHAAr, |
| | | | (CH₂)ₘCHAHet or (CH₂)ₘCHA-cycloalkyl, |
| | | | in which |
| | | Ar | is phenyl, naphthyl, anthranyl or fluorenyl, |
| | | | each of which is unsubstituted or monosubstituted |
| | | | or polysubstituted by Hal, A, OA, OH, |
| | | | CO-A, CN, COOA, COOH, CONH₂, CONHA, |
| | | | CONA₂, CF₃, OCF₃ or NO₂, |
| | | | and in which, in the presence of |
| | | | (CH₂)ₘCHAAr, (CH₂)ₘCHAHet or (CH₂)ₘCHA- |
| | | | cycloalkyl, m = 0; |
| in | lc) | R² | is H, A, alkenyl, (CH₂)ₘAr, (CH₂)ₘHet, |
| | | | (CH₂)ₘcycloalkyl or CHAAr; |
| in | ld) | R¹, R^{1′} | are H, Ar, Het, Hal, OA, NHA, NA₂, COOA, |
| | | and R^{1″} | CONH₂, CONHA and/or CONA₂ |
| in | le) | R¹ | is Ar |
| in | lf) | R¹ | is Ar, |
| | | | in which |
| | | Ar | is a phenyl radical, which is unsubstituted or |
| | | | monosubstituted or disubstitiuted by A, OA, |
| | | | OH, Hal or CF₃ |
| in | lg) | R¹ | is Ar, |
| | | | in which |
| | | Ar | is phenyl |
| | | R^{1′} and | are each H, |
| | | R^{1″} | |
| in | ih) | X | is O or S |
| | | Y | independently of one another are NH or O |
| | | R¹, R^{1′} | are H, A, Ar, Het, Hal, OA, NH₂, NHA, NA₂, |
| | | and R^{1″} | COOA, CONH₂, CONHA, CONA₂ and/or |
| | | | alkenyl having from 1 to 4 carbon atoms and |
| | | | 1 double bond |
| | | R² | is H, A, (CH₂)ₘAr, (CH₂)ₘHet, (CH₂)ₘcycloalkyl |
| | | | or CHAAr |
| | | A | is alkyl having from 1 to 6 carbon atoms |
| | | Ar | is phenyl, naphthyl or biphenyl, each of which |
| | | | is unsubstituted or monosubstituted or disubstitiuted |
| | | | by Hal, A, OA, COOA, COOH, |
| | | | CONH₂, CONHA, CONA₂, CF₃, or OCF₃ |
| | | Het | is an aromatic monocyclic or bicyclic hetero- |
| | | | cyclic radical having from 1 to 4 N, O and/or S |
| | | | atoms, which may be unsubstituted or |
| | | | monosubstituted or disubstituted by Hal, A, OA, |
| | | | OCF₃, —CO-A, COOA, CONH₂, CONHA, |
| | | | CONA₂, NH₂, NHA, or NA₂ |
| | | m | is 0, 1 or 2 |
| | | n | is 2 or 3, |
| in | ii) | X | is O or S |
| | | Y | independently of one another are NH or O |
| | | R¹ | is Ar¹ |
| | | R^{1′} and | are each H, |
| | | R^{1″} R² | is H, A, (CH₂)ₘAr², (CH₂)ₘcycloalkyl, alkenyl |
| | | | having from 1 to 4 carbon atoms and 1 double |
| | | | bond, or CHA′Ar², |
| | | | in which A′ is alkyl having 1, 2 or 3 carbon |
| | | | atoms, |
| | | A | is alkyl having from 1 to 6 carbon atoms, |
| | | Ar¹ | is phenyl |
| | | Ar² | is phenyl, naphthyl or fluorenyl, each of which |
| | | | is unsubstituted or monosubstituted or disubstitiuted |
| | | | by Hal, A, OA, COOA, CONH₂, |
| | | | CONHA, CONA₂, CF₃, or OCF₃ |
| | | m | is 0, 1 or 2 |
| | | n | is 1, 2 or 3. |

Particular preference is given to the compounds of the general formula I mentioned below:

3-biphenyl-4-yl-3-{2-[3-(3-benzylthioureido)propanoylamino]ethanoylamino}propionic acid,

3-biphenyl-4-yl-3-{2-[4-(3-benzylureido)butanoylamino]ethanoylamino}propionic acid,

3-biphenyl-4-yl-3-[2-(4-ureidobutanoylamino)ethanoylamino}propionic acid,

3-biphenyl-4-yl-3-{2-[4-(3-ethylureido)butanoylamino]ethanoylamino}-propionic acid,

3-biphenyl-4-yl-3-{2-[4-(3-cyclohexylureido)butanoylamino]ethanoylamino}-propionic acid,

3-biphenyl-4-yl-3-{2-[4-(3-isoproylureido)butanoylamino]ethanoylamino}-propionic acid,

3-biphenyl-4-yl-3-{2-[4-(3-butylureido)butanoylamino]ethanoylamino}propionic acid,

3-biphenyl-4-yl-3-{2-[4-(3-tert-butylureido)butanoylamino]ethanoylamino}-propionic acid,

3-biphenyl-4-yl-3-{2-[4-(3-methylthioureido)butanoylamino]ethanoylamino}-propionic acid,

3-biphenyl-4-yl-3-{2-[4-(3-methylthioureido)butanoylamino]ethanoylamino}-propionic acid,

3-biphenyl-4-yl-3-{2-[4-(3-phenylureido)butanoylamino]ethanoylamino}-propionic acid,

3-biphenyl-4-yl-3-{2-[4-(3-phenylethylureido)butanoylamino]ethanoylamino}-propionic acid,

3-biphenyl-4-yl-3-{2-[4-(3-(2-chlorophenyl)ureido)butanoylamino]ethanoylamino} propionic acid,

3-biphenyl-4-yl-3-{2-[4-(3-(3-chlorophenyl)ureido)butanoylamino]ethanoylamino} propionic acid,

3-biphenyl-4-yl-3-{2-[4-(3-(4-chlorophenyl)ureido)butanoylamino]ethanoylamino} propionic acid,

3-biphenyl-4-yl-3-{2-[4-(3-(2-methoxyphenyl)ureido)butanoylamino]-ethanoylamino} propionic acid,

3-biphenyl-4-yl-3-{2-[4-(3-(4-methoxyphenyl)ureido)butanoylamino]-ethanoylamino} propionic acid,

3-biphenyl-4-yl-3-[2-(3-ureidopropanoylamino)ethanoylamino]propionic acid,

3-biphenyl-4-yl-3-{2-[3-(3-ethylureido)propanoylamino]ethanoylamino}-propionic acid,

3-biphenyl-4-yl-3-{2-[3-(3-cyclohexylureido)propanoylamino]ethanoylamino}propionic acid,

3-biphenyl-4-yl-3-{2-[3-(3-butylureido)propanoylamino]ethanoylamino}propionic acid,

3-biphenyl-4-yl-3-{2-[3-(3-tert-butylureido)propanoylamino]ethanoylamino}-propionic acid,

3-biphenyl-4-yl-3-{2-[3-(3-methylthioureido)propanoylamino]ethanoylamino}-propionic acid,

3-biphenyl-4-yl-3-{2-[3-(3-phenylureido)propanoylamino]ethanoylamino}propionic acid,

3-biphenyl-4-yl-3-{2-[3-(3-phenylethylureido)propanoylamino]ethanoylamino}propionic acid,

3-biphenyl-4-yl-3-{2-[3-(3-(2-chlorophenylethyl)ureido)propanoylamino]-ethanoylamino}propionic acid,

3-biphenyl-4-yl-3-{2-[3-(3-(3-chlorophenylethyl)ureido)propanoylamino]-ethanoylamino}propionic acid,

3-biphenyl-4-yl-3-{2-[3-(3-(4-chlorophenylethyl)ureido)propanoylamino]-ethanoylamino}propionic acid,

3-biphenyl-4-yl-3-{2-[3-(3-(2-methoxyphenyl)ureido)propanoylamino]-ethanoylamino}propionic acid,

3-biphenyl-4-yl-3-{2-[3-(3-(4-methoxyphenyl)ureido)propanoylamino]-ethanoylamino}propionic acid,

3-biphenyl-4-yl-3-{2-[3-(3-(3-methoxyphenyl)ureido)propanoylamino]-ethanoylamino}propionic acid,

3-biphenyl-4-yl-3-{2-[3-(3-((R)-1-phenylethyl)ureido)propanoylamino]-ethanoylamino}propionic acid,

3-biphenyl-4-yl-3-{2-[3-(3-((S)-1-phenylethyl)ureido)propanoylamino]-ethanoylamino}propionic acid,

3-biphenyl-4-yl-3-{2-[3-(3-((R)-1-naphthalen-1-ylethyl)ureido)propanoylamino]ethanoylamino}propionic acid,

3-biphenyl-4-yl-3-{2-[3-(3-((S)-1-naphthalen-1-ylethyl)ureido)propanoylamino]ethanoylamino}propionic acid,

3-biphenyl-4-yl-3-{2-[3-(3-naphthalen-1-ylureido)propanoylamino]ethanoylamino}propionic acid,

3-biphenyl-4-yl-3-{2-[3-(3-naphthalen-2-ylureido)propanoylamino]ethanoylamino}propionic acid,

3-biphenyl-4-yl-3-{2-[3-(3-benzylthioureido)propanoylamino]ethanoylamino}propionic acid,

3-biphenyl-4-yl-3-{2-[3-(3-(4-methylbenzyl)ureido)propanoylamino]ethanoylamino}propionic acid,

3-biphenyl-4-yl-3-{2-[3-(3-(2,4-dichlorobenzyl)ureido)propanoylamino]-ethanoylamino}propionic acid,

3-biphenyl-4-yl-3-{2-[3-(3-(4-fluorobenzyl)ureido)propanoylamino]ethanoyl-amino}propionic acid,

3-biphenyl-4-yl-3-{2-[3-(3-(3,4-dichlorobenzyl)ureido)propanoylamino]ethanoylamino}propionic acid,

3-biphenyl-4-yl-3-{2-[3-(3-(2-chlorobenzyl)ureido)propanoylamino]ethanoyl-amino}propionic acid,

3-biphenyl-4-yl-3-{2-[3-(3-(3-propyl)ureido)propanoylamino]ethanoylamino}-propionic acid,

3-biphenyl-4-yl-3-{2-[3-(3-allylureido)propanoylamino]ethanoylamino}-propionic acid,

3-biphenyl-4-yl-3-[2-(5-ureidopentanoylamino)ethanoylamino]propionic acid,

3-biphenyl-4-yl-3-{2-15-(3-ethylureido)pentanoylamino]ethanoylamino}propionic acid,

3-biphenyl-4-yl-3-{2-[5-(3-cyclohexylureido)pentanoylamino]ethanoyl-amino}propionic acid,

3-biphenyl-4-yl-3-{2-[5-(3-isopropylureido)pentanoylamino]ethanoylamino}propionic acid,

3-biphenyl-4-yl-3-{2-[5-(3-butylureido)pentanoylamino]ethanoylamino}propionic acid,

3-biphenyl-4-yl-3-{2-[5-(3-tert-butylureido)pentanoylamino]ethanoylamino}propionic acid,

3-biphenyl-4-yl-3-{2-[5-(3-methylthioureido)pentanoylamino]ethanoyl-amino}propionic acid,

3-biphenyl-4-yl-3-{2-[5-(3-phenylureido) pentanoylamino]ethanoylamino}propionic acid,

3-biphenyl-4-yl-3-{2-[5-(3-phenylethylureido)pentanoylamino]ethanoyl-amino}propionic acid,

3-biphenyl-4-yl-3-{2-[5-(3-(2-chlorophenyl)ureido)pentanoylamino]ethanoyl-amino}propionic acid,

3-biphenyl-4-yl-3-{2-[5-(3-(3-chlorophenyl)ureido)pentanoylamino]ethanoyl-amino}propionic acid,

3-biphenyl-4-yl-3-{2-[5-(3-(4-chlorophenyl)ureido)pentanoylamino]ethanoyl-amino}propionic acid,

3-biphenyl-4-yl-3-{2-[5-(3-(2-methoxyphenyl)ureido)pentanoylamino]-ethanoylamino}propionic acid,

3-biphenyl-4-yl-3-{2-[5-(3-(4-methoxyphenyl)ureido)pentanoylamino]-ethanoylamino}propionic acid,

3-biphenyl-4-yl-3-{2-[5-(3-(3-methoxy-phenyl)ureido)pentanoylamino]-ethanoylamino}propionic acid,

3-biphenyl-4-yl-3-{2-[4-(3-((R)-1-phenylethyl)ureido)butanoylamino]-ethanoylamino}propionic acid,

3-biphenyl-4-yl-3-{2-[4-(3-((S)-1-phenylethyl)ureido)butanoylamino]-ethanoylamino}propionic acid,

3-biphenyl-4-yl-3-{2-[4-(3-naphthalen-1-ylureido)butanoylamino]ethanoyl-amino}propionic acid,

3-biphenyl-4-yl-3-{2-[4-(3-benzylthioureido)butanoylamino]ethanoylamino}propionic acid,

3-biphenyl-4-yl-3-{2-[4-(3-naphthalen-2-ylureido)butanoylamino]ethanoyl-amino}propionic acid,

3-biphenyl-4-yl-3-{2-[4-(3-(4-methylbenzyl)ureido)butanoylamino]ethanoylamino}propionic acid,

3-biphenyl-4-yl-3-{2-[4-(3-(3-methylbenzyl)ureido)butanoylamino]ethanoyl-amino}propionic acid,

3-biphenyl-4-yl-3-{2-[4-(3-(2-methylbenzyl)ureido)butanoylamino]ethanoyl-amino}propionic acid,

3-biphenyl-4-yl-3-{2-[4-(3-(2,4-dichlorobenzyl)ureido)butanoylamino]-ethanoylamino}propionic acid,

3-biphenyl-4-yl-3-{2-[4-(3-(4-fluorobenzyl)ureido)butanoylamino]ethanoyl-amino}propionic acid,

3-biphenyl-4-yl-3-{2-[4-(3-(3,4-dichlorobenzyl)ureido)butanoylamino]-ethanoylamino}propionic acid,

3-biphenyl-4-yl-3-{2 -[4-(3-(2-chlorobenzyl)ureido)butanoylamino]ethanoyl-amino}propionic acid,

3-biphenyl-4-yl-3-{2-[4-(3-(4-methoxybenzyl)ureido)butanoylamino]-ethanoylamino}propionic acid,

3-biphenyl-4-yl-3-{2-[4-(3-propylureido)butanoylamino]ethanoylamino}-propionic acid,

3-biphenyl-4-yl-3-{2-[4-(3-allylureido)butanoylamino]ethanoylamino}propionic acid,

3-biphenyl-4-yl-3-{2-[5-(3-((R)-1-phenylethyl)ureido)pentanoylamino]-ethanoylamino}propionic acid,

3-biphenyl-4-yl-3-{2-[5-(3-((S)-1-phenylethyl)ureido)pentanoylamino]-ethanoylamino}propionic acid,

3-biphenyl-4-yl-3-{2-[5-(3-naphthalen-1-ylureido)pentanoylamino]ethanoylamino}propionic acid,

3-biphenyl-4-yl-3-{2-[5-(3-benzylthiou reido)pentanoylamino]ethanoylamino}propionic acid,

3-biphenyl-4-yl-3-{2-[5-(3-naphthalen-2-ylureido)pentanoylamino]ethanoylamino}propionic acid,

3-biphenyl-4-yl-3-{2-[5-(3-(4-methylbenzyl)ureido)pentaoylamino]ethanoylamino}propionic acid,

3-biphenyl-4-yl-3-{2-[5-(3-(3-methylbenzyl)ureido)pentanoylamino]ethanoylamino}propionic acid,

3-biphenyl-4-yl-3-{2-[5-(3-(2-methylbenzyl)ureido)pentanoylamino]ethanoylamino}propionic acid,

3-biphenyl-4-yl-3-{2-[5-(3-(2,4-dichlorobenzyl)ureido)pentanoylamino]-ethanoylamino}propionic acid,

3-biphenyl-4-yl-3-{2-[5-(3-(4-fluorobenzyl)ureido)pentanoylamino]ethanoylamino}propionic acid,

3-biphenyl-4-yl-3-{2-[5-(3-(3,4-dichlorobenzyl)ureido)pentanoylamino]-ethanoylamino}propionic acid,

3-biphenyl-4-yl-3-{2-[5-(3-(2-chlorobenzyl)ureido)pentanoylamino]ethanoylamino}propionic acid,

3-biphenyl-4-yl-3-{2-[5-(3-(4-methoxybenzyl)ureido)pentanoylamino]-ethanoylamino}propionic acid,

3-biphenyl-4-yl-3-{2-[5-(3-propylureido)pentanoylamino]ethanoylamino}-propionic acid,

3-biphenyl-4-yl-3-{2-[3-(9H-fluoren-9-ylmethoxycarbonylamino)propanoylamino]ethanoylamino}propionic acid,

3-biphenyl-4-yl-3-{2-[4-(9H-fluoren-9-ylmethoxycarbonylamino)butanoyl-amino]ethanoylamino}propionic acid,

3-biphenyl-4-yl-3-{2-[5-(9H-fluoren-9-ylmethoxycarbonylamino)pentanoyl-amino]ethanoylamino}propionic acid

3-biphenyl-4-yl-3-{2-[5-(3-allylureido)pentanoylamino]ethanoylamino}-propionic acid,

3-biphenyl-4-yl-3-{2-[3-(3-ethoxycarbonylamino)propanoylamino]ethanoyl-amino}propionic acid,

3-biphenyl-4-yl-3-{2-[3-(3-benzyloxycarbonylamino)propanoylamino]-ethanoylamino}propionic acid,

3-biphenyl-4-yl-3-{2-[3-(3-(2,2-dimethylpropoxycarbonylamino)propanoylamino]ethanoylamino}propionic acid,

3-biphenyl-4-yl-3-{2-[3-(4-ethyloxycarbonylaminobutanoylamino]ethanoylamino}propionic acid,

3-biphenol-4-yl-3-{2-[3-(4-benzyloxycarbonylaminobutanoylamino]ethanoylamino}propionic acid,

3-biphenyl-4-yl-3-{2-[3-(4-(2,2-dimethylpropyloxycarbonylaminobutanoylamino]ethanoylamino}propionic acid,

3-biphenyl-4-yl-3-{2-[3-(9H-fluoren-9-ylmethoxycarbonylamino)propanoylamino]ethanoylamino}propionic acid,

3-biphenyl-4-yl-3-{2-[4-(9H-fluoren-9-ylmethoxycarbonylamino)butanoylamino]ethanoylamino}propionic acid,

3-biphenyl-4-yl-3-{2-[5-(9H-fluoren-9-ylmethoxycarbonylamino)pentanoylamino]ethanoylamino}propionic acid,

3-biphenyl-4-yl-3-[2-(3-ethoxycarbonylaminopropanoylamino)ethanoylamino]propionic acid,

3-biphenyl-4-yl-3-[2-(3-benzyloxycarbonylaminopropanoylamino)ethanoylamino]propionic acid,

3-biphenyl-4-yl-3-{2-[3-(2,2-dimethylpropoxycarbonylamino)propanoylamino]ethanoylamino}propionic acid,

3-biphenyl-4-yl-3-{2-[4-ethoxycarbonylaminobutanoylamino]ethanoylamino}propionic acid,

3-biphenyl-4-yl-3-[2-(4-benzyloxycarbonylamino-butanoylamino)ethanoylamino]propionic acid,

3-biphenyl-4-yl-3-{2-[4-(2,2-dimethylpropoxycarbonylamino)butanoylamino]-ethanoylamino}propionic acid.

The compounds of the formula I according to claim 1 and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for said reactions. Use can also be made here of variants which are known per se, but are not mentioned here in greater detail.

If desired, the starting materials can also be formed in situ, so that they are not isolated from the reaction mixture, but are instead immediately converted further into the compounds of the formula I according to claim 1.

It is also possible for a plurality of—identical or different—protected amino and/or hydroxyl groups to be present in the molecule of the starting material. If the protecting groups present differ from one another, they can in many cases be removed selectively (cf. in this respect: T. W. Greene, P. G. M. Wuts, Protective Groups in Organic Chemistry, 2nd Edn., Wiley, New York 1991 or P. J. Kocienski, Protecting Groups, 1st Edn., Georg Thieme Verlag, Stuttgart -New-York, 1994).

The term “amino protecting group” is generally known and relates to groups which are suitable for protecting (blocking) an amino group against chemical reactions. Typical of such groups are, in particular, unsubstituted or substituted acyl, aryl, aralkoxymethyl or aralkyl groups. Since the amino protecting groups are removed after the desired reaction (or synthesis sequence), their type and size is furthermore not crucial; however, preference is given to those having 1-20, in particular 1-8 carbon atoms. The term “acyl group” is to be understood in the broadest sense in connection with the present process. It includes acyl groups derived aliphatic, araliphatic, alicyclic, aromatic and heterocyclic carboxylic acids or sulfonic acids, as well as, in particular, alkoxycarbonyl, alkenyloxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as phenoxyacetyl; alkoxycarbonyl, such as methoxycarbonyl, ethoxycarbonyl, 2,2,2-trichloroethoxycarbonyl, BOC and 2-iodoethoxycarbonyl; alkenyloxy-carbonyl, such as allyloxycarbonyl (Aloc), aralkoxycarbonyl, such as CBZ (synonymous with Z), 4-methoxybenzyloxycarbonyl (MOZ), 4-nitrobenzyl-oxycarbonyl and 9-fluorenylmethoxycarbonyl (Fmoc); 2-(phenylsulfonyl)-ethoxycarbonyl; trimethylsilylethoxycarbonyl (Teoc), and arylsulfonyl, such as 4-methoxy-2,3,6-trimethylphenylsulfonyl (Mtr). Preferred amino protect-ing groups are BOC, Fmoc and Aloc, furthermore CBZ, benzyl and acetyl. Particularly preferred protecting groups are BOC and Fmoc.

The term “hydroxyl protecting group” is likewise generally known and relates to groups which are suitable for protecting a hydroxyl group against chemical reactions. Typical of such groups are the above-mentioned unsubstituted or substituted aryl, aralkyl, aroyl or acyl groups, furthermore also alkyl groups, alkyl-, aryl- and aralkylsilyl groups, and O,O- and O,S-acetals. The nature and size of the hydroxyl protecting groups is not crucial since they are removed again after the desired chemical reaction or synthesis sequence; preference is given to groups having 1-20 carbon atoms, in particular 1-10 carbon atoms. Examples of hydroxyl protecting groups are, inter alia, aralkyl groups, such as benzyl, 4-methoxybenzyl and 2,4-di-methoxybenzyl, aroyl groups, such as benzoyl and p-nitrobenzoyl, acyl groups, such as acetyl and pivaloyl, p-toluenesulfonyl, alkyl groups, such as methyl and tert-butyl, but also allyl, alkylsilyl groups, such as trimethyl-silyl (TMS), triisopropylsilyl (TIPS), tert-butyidimethylsilyl (TBS) and triethyl-silyl, trimethylsilylethyl, aralkylsilyl groups, such as tert-butyldiphenylsilyl (TBDPS), cyclic acetals, such as isopropylidene acetal, cyclopentylidene acetal, cyclohexylidene acetal, benzylidene acetal, p-methoxybenzylidene acetal and o,p-dimethoxybenzylidene acetal, acyclic acetals, such as tetrahydropyranyl (Thp), methoxymethyl (MOM), methoxyethoxymethyl (MEM), benzyloxymethyl (BOM) and methylthiomethyl (MTM). Particularly preferred hydroxyl protecting groups are benzyl, acetyl, tert-butyl and TBS.

The liberation of the compounds of the formula I from their functional derivatives is known from the literature for the protecting group used in each case (for example T. W. Greene, P. G. M. Wuts, Protective Groups in Organic Chemistry, 2nd Edn., Wiley, New York 1991 or P. J. Kocienski, Protecting Groups, 1st Edn., Georg Thieme Verlag, Stuttgart -New York, 1994). Use may also be made here of variants which are known per se, but are not mentioned here in greater detail.

The groups BOC and O-tert-butyl may, for example, be removed preferentially using TFA in dichloromethane or using approximately 3 to 5N HCl in dioxane at 15-30° C., and the Fmoc group using an approximately 5 to 50% solution of dimethylamine, diethylamine or piperidine in DMF at 15-30° C. The Aloc group can be removed under gentle conditions with noble-metal catalysis in chloroform at 20-30° C. A preferred catalyst is tetrakis(triphenyl-phosphine)palladium(0).

The starting compounds of the formulae II to VI and 1 to 3 are generally known. If they are novel, however, they can be prepared by methods known per se.

The compounds of the formula I can also be synthesised on a solid phase, the binding to the solid phase taking place via the OH of the carboxyl group. In the case of synthesis on a solid phase, the carboxyl group is substituted by OPol, where Pol is a solid phase without a terminal functional group. Pol represents the polymeric support material and all atoms of the anchor group of a solid phase apart from the terminal functional group. The anchor groups of a solid phase, also known as linkers, are necessary for binding of the compound to be functionalised to the solid phase. A review of syntheses on the solid phase and the solid phases and/or linkers which can be employed for this purpose is given, for example, in Novabiochem-The Combinatorial Chemistry Catalog, March 99, pages S1-S72.

Particularly suitable solid phases for the synthesis of compounds according to the invention are solid phases having a hydroxyl group as terminal functionality, for example Wang resin or polystyrene A OH.

Compounds of the formula II with R¹═Ar and R═OL, where L is Pol, are prepared, for example, in accordance with the following reaction scheme 1, where SG, denotes an amino-protecting group, as described above.

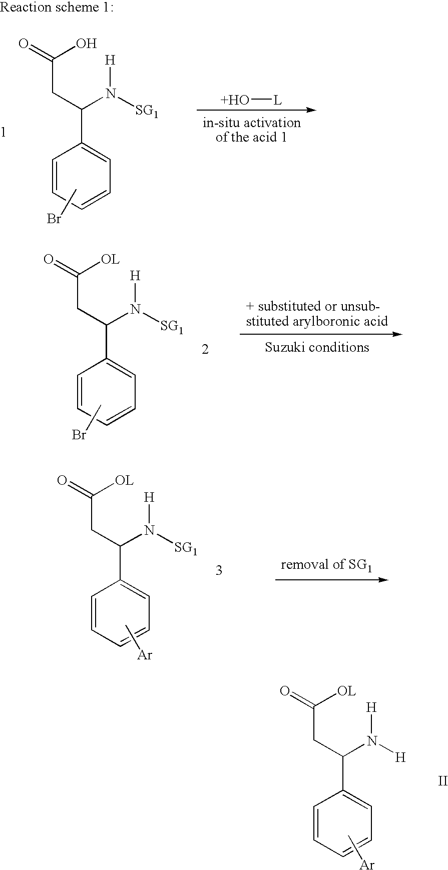

The bromophenyl-substituted carboxylic acid 1 is activated in situ by known methods, for example by reaction with diisopropylcarbodiimide, and reacted with the alcohol HO-L, where L is as defined above. The subsequent coupling of compound 2 to an unsubstituted or substituted arylboronic acid under Suzuki conditions generates the derivative 3. The removal of the protecting group SG₁ under known conditions liberates a compound of the formula II.

The Suzuki reaction is advantageously carried out with palladium control, preferably by addition of Pd(PPh₃)₄, in the presence of a base, such as potassium carbonate, in an inert solvent or solvent mixture, for example DMF, at temperatures between 0° and 150°, preferably between 60° and 120°. The reaction time, depending on the conditions used, is between a few minutes and several days. The boronic acid derivatives can be prepared by conventional methods or are commercially available. The reactions can be carried out analogously to the methods indicated in Suzuki et al., J. Am. Chem. Soc. 1989, 111, 314 ff and in Suzuki et al. Chem. Rev. 1995, 95, 2457 ff.

Compounds of the formula I are obtained by peptide-analogous coupling of the compounds of the formula II with a compound of the formula III or by peptide-analogous coupling of the compounds of the formula IV with a compound of the formula V under standard conditions.

Compounds of the formula III are obtained by peptide-analogous coupling of the compounds of the formula V with an amino compound H₂N—CH₂—COOSG² under standard conditions, where SG² denotes a hydroxyl protecting group, as described above, which is removed after the coupling. Compounds of the formula IV are obtained by peptide-analogous coupling of a compound of the formula II with a carboxyl compound HOOC—CH₂—NHSG₁ under standard conditions, where SG₁ is an amino-protecting group as described above which is cleaved off after the coupling. Conventional methods of peptide synthesis are described, for example, in Houben-Weyl, 1.c., Volume 15/II, 1974, pages 1 to 806.

The coupling reaction preferably succeeds in the presence of a dehydrating agent, for example a carbodiimide, such as dicyclohexylcarbodiimide (DCC), N-(3-dimethylaminopropyl)-N′-ethylcarbodiimide hydrochloride (EDC) or diisopropylcarbodiimide (DIC), furthermore, for example, propane-phosphonic anhydride (cf. Angew. Chem. 1980, 92, 129), diphenylphosphoryl azide or 2-ethoxy-N-ethoxycarbonyl-1,2-dihydroquinoline, in an inert solvent, for example a halogenated hydrocarbon, such as dichloromethane, an ether, such as tetrahydrofuran or dioxane, an amide, such as DMF or dimethylacetamide, a nitrile, such as acetonitrile, in dimethyl sulfoxide or in the presence of this solvent, at temperatures between about −10 and 40°, preferably between 0 and 30°. The reaction time, depending on the conditions used, is between a few minutes and several days.

It has proven particularly advantageous to add the coupling reagent TBTU (O-(benzotriazol-1-yl)-N,N,N′,N′-tetramethyluronium tetrafluoroborate) or O-(benzotriazol-1-yl)-N,N,N′,N′-tetramethyluronium hexafluorophosphate, since in the presence of one of these compounds only slight racemisation occurs and no cytotoxic by-products are formed.

Instead of compounds of the formula II, V and/or VI, it is also possible to employ derivatives of compounds of the formula III, VI and/or VI, preferably a pre-activated carboxylic acid, or a carboxylic acid halide, a symmetrical or mixed anhydride or an active ester. Radicals of this type for activation of the carboxyl group in typical acylation reactions have been described in the literature (for example in the standard works, such as Houben-Weyl, Methoden der organischen Chemie [Methods of Organic Chemistry], Georg-Thieme-Verlag, Stuttgart). Activated esters are advantageously formed in situ, for example by addition of HOBt (1-hydroxybenzotriazole) or N-hydroxysuccinimide.

The reaction is generally carried out in an inert solvent; if a carboxylic acid halide is used, it is carried out in the presence of an acid-binding agent, preferably an organic base, such as triethylamine, dimethylaniline, pyridine or quinoline.

The addition of an alkali or alkaline-earth metal hydroxide, carbonate or bicarbonate or another salt of a weak acid of the alkali or alkaline-earth metals, preferably of potassium, sodium, calcium or caesium, may also be favourable.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, sulfurous acid, dithionic acid, nitric acid, hydro-halic acids, such as hydrochloric acid or hydrobromic acid, phosphoric acids, such as, for example, orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, benzenesulfonic acid, trimethoxybenzoic acid, adamantanecarboxylic acid, p-toluenesulfonic acid, glycolic acid, embonic acid, chlorophenoxy-acetic acid, aspartic acid, glutamic acid, proline, glyoxylic acid, palmitic acid, para-chlorophenoxyisobutyric acid, cyclohexanecarboxylic acid, glucose 1-phosphate, naphthalenemono- and -disulfonic acids or lauryl-sulfuric acid. Salts with physiologically unacceptable acids, for example picrates, can be used to isolate and/or purify the compounds of the formula I.

On the other hand, compounds of the formula I can be converted into the corresponding metal salts, in particular alkali metal salts or alkaline earth metal salts, or into the corresponding ammonium salts, using bases (for example sodium hydroxide, potassium hydroxide, sodium carbonate or potassium carbonate).

The invention also relates to the compounds of the formula I according to claim 1, their stereoisomers and/or their physiologically acceptable salts or solvates as medicament active ingredients.

The invention furthermore relates to compounds of the formula I according to claim 1, their stereoisomers and/or their physiologically acceptable salts or solvates as integrin agonists and/or antagonists.

The invention also relates to the compounds of the formula I according to claim 1, their stereoisomers and/or their physiologically acceptable salts or solvates for use in combating illnesses. The use of the compounds for combating illnesses covers their use for therapy and/or prophylaxis.

The invention furthermore relates to pharmaceutical preparations at least comprising a compound of the formula I according to claim 1 or 2, their stereoisomers and/or a physiologically acceptable salt or solvates thereof. The compounds of the formula I can be brought into a suitable dosage form here together with at least one solid, liquid and/or semiliquid excipient or assistant and, if desired, in combination with one or more further active ingredients.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oily or aqueous solutions, furthermore suspensions, emulsions or implants, and suitable for topical application are ointments, creams or powders. The novel compounds can also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, dyes, flavours and/or a plurality of further active ingredients, for example one or more vitamins.

For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronised form, in which case one or more additional physiologically acceptable solvents may be present, for example ethanol. Inhalation solutions can be administered with the aid of conventional inhalers.

The compounds of the formula I, their stereoisomers and/or their physiologically acceptable salts or solvates can be employed as medicament active ingredients in human and veterinary medicine, in particular for the prophylaxis and/or therapy of circulation disorders, pulmonary fibrosis, pulmonary embolism, thrombosis, in particular deep-vein thrombosis, cardiac infarction, arteriosclerosis, aneurysma dissecans, transient is chaemic attacks, apoplexia, angina pectoris, in particular unstable angina pectoris, pathological connecting tissue proliferation in organs or fibrosis, particular pulmonary fibrosis, but also cystic fibrosis, dermatofibrosis, hepatic fibrosis, liver cirrhosis, urethrofibrosis, renal fibrosis, cardiac fibrosis, infantile endocardial fibrosis, pancreatic fibrosis, disturbed hornification of the skin, in particular leukoplakia, lichen planus and squamous cell carcinoma, tumour illnesses, such as tumour development, tumour angiogenesis or tumour metastasis, of solid tumours and those of the blood or immune system, for example tumours of the skin, squamous cell carcinoma, tumours of the blood vessels, of the gastro-intestinal tract, of the lung, of the breast, of the liver, of the kidney, of the spleen, of the pancreas, of the brain, of the testes, of the ovary, of the womb, of the vagina, of the muscles, of the bones, and those of the throat and head area, osteolytic illnesses, such as osteoporosis, hyperparathyroidism, Paget's disease, malign hypercalcaemia, incompatible blood transfusion, pathologically angiogenic disorders, such as, for example, inflammation, ophthalmological disorders, diabetic retinopathy, macular degeneration, myopia, corneal transplant, ocular histoplasmosis, rheumatic arthritis, osteoarthritis, rubeotic glaucoma, ulcerative colitis, Crohn's disease, atherosclerosis, psoriasis, restenosis, in particular after angioplasty, multiple sclerosis, pregnancy, absumptio placentaris, viral infection, bacterial infection, fungal infection, foot and mouth disease (FMD), HIV, anthrax, candida albicans, in the case of parasitic infestation, in the case of acute kidney failure and in the case of wound healing for supporting the healing process.

In the case of viral infection, the compounds according to the invention act, in particular, by inhibiting or breaking viral bonds between cell-mediated integrin-binding proteins and the viral shell or indirectly by preventing the uptake of the viruses, which are bound to extracellular matrix constituents, which have been recognised as integrins, or by breaking integrin-promoted mechanisms which are associated with the viral infection (J Virol 2000 June;74(II):4949-56, J Virol 2000 August;74(16):7298-306, J Virol 2001 May;75(9):4158-64, Virology. 2001 Sep. 30;288(2):192-202. (FMDV), Virus Res. 2001 July;76(1):1-8 (echovirus), J Biol. Chem. 2001 Jul. 13;276(28):26204-10. (HIV), Biochem Biophys Res Commun. 2001 May 11 ;283(3):668-73 (papillomavirus), Proc Natl Acad Sci USA. 2000 Dec. 19;97(26): 14644-9 (rotavirus)).

In the case of bacterial infection, the action takes place, in particular, by inhibition of the binding and/or the uptake of the bacteria or bacterial toxins or of the toxic products induced by bacterial infections to or by cells via integrin-promoted mechanisms (Nature 2001: Nov 8:225-229 (anthrax), J Exp Med. 2001 May 7;193(9):103544 (pertussis), Proc Natl Acad Sci U S A. 2000 Feb. 29;97(5):223540 (group A streptococcus), Infect Immun. 2000 January;68(1):72-9 (Pasteurella haemolytica leucotoxin), J Biol. Chem. 1997 Nov. 28;272(48):30463-9. (RTX leucotoxins)).

In the case of parasitic infestation, the action takes place, in particular, by inhibition of the binding and/or uptake of the parasitic or parasite-derived or induced toxins to or by the cells via integrin-directed mechanisms (Infect Immun. 1999 September;67(9):4477-84.(Ieishmania)).

The substances according to the invention are generally preferably administered in doses of from about 0.05 to 500 mg, in particular from 0.5 to 100 mg, per dosage unit. The daily dose is preferably from about 0.01 to 2 mg/kg of body weight. However, the specific dose for each patient depends on a wide variety of factors, for example on the efficacy of the specific compound employed, on the age, body weight, general state of health, sex, on the diet, on the time and method of administration, on the rate of excretion, medicament combination and severity of the particular illness to which the therapy applies. Parenteral administration is preferred.

Furthermore, the compounds of the formula I can be used as integrin ligands for the production of columns for affinity chromatography for the purification of integrins.

In this method, the ligand, i.e. a compound of the formula I, is covalently coupled to a polymeric support via an anchor function, for example the carboxyl group.

Suitable polymeric support materials are the polymeric solid phases having preferably hydrophilic properties that are known in peptide chemistry, for example crosslinked polysugars, such as cellulose, sepharose or Sephadex^{R}, acrylamides, polyethylene glycol-based polymers or Tentakel^{R} polymers.

The materials for affinity chromatography for integrin purification are prepared under conditions as are usual and known, per se for the condensation of amino acids.

The compounds of the formula I have one or more centres of chirality and can therefore exist in racemic or optically active form. Racemates obtained can be resolved into the enantiomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid, and the various optically active camphorsulfonic acids, such as β-camphorsulfonic acid. Resolution of the enantiomers with the aid of a column filled with an optically active resolving agent (for example dinitrobenzoylphenylglycine) is also advantageous; an example of a suitable eluent is a mixture of hexane/isopropanol/acetonitrile, for example in the volume ratio 82:15:3.

It is of course also possible to obtain optically active compounds of the formula I by the methods described above by using starting materials which are already optically active.

Above and below, all temperatures are given in ° C. In the following examples, “conventional work-up” means that, if necessary, water is added, if necessary, depending on the constitution of the end product, the pH is adjusted to a value between 2 and 10, the mixture is extracted with ethyl acetate or dichloromethane, the phases are separated, the organic phase is dried over sodium sulfate and evaporated, and the product is purified by chromatography on silica gel, by preparative HPLC and/or by crystallisation. The purified compounds are, if desired, freeze-dried.

The eluents used are gradients of acetonitrile (B) with 0.08% of TFA (tri-fluoroacetic acid) and water (A) with 0.1% of TFA. The gradient is indicated in percent by volume of acetonitrile.

The HPLC analyses (retention time RT) were carried out in the following systems:

3 μm Silica-Rod column with a 210 second gradient from 20 to 100% water/acetonitrile/0.01% trifluoroacetic acid, at a flow rate of 2.2 ml/min and with detection at 220 nm.

The compounds purified by preparative HPLC are isolated as trifluoro-acetates.

Mass spectrometry (MS) by means of FAB (fast atom bombardment): MS-FAB (M+H)⁺.

The examples explain the invention without the latter being restricted thereto. If the compounds described as examples are able to exist as various stereoisomers and no stereochemical data are given, mixtures of the stereoisomers are present in each case.

### EXAMPLE 1

Synthesis of 3-biphenyl-4-yl-3-{2-[3-(3-isopropylureido)propanoylamino]-ethanoylamino}propionic acid

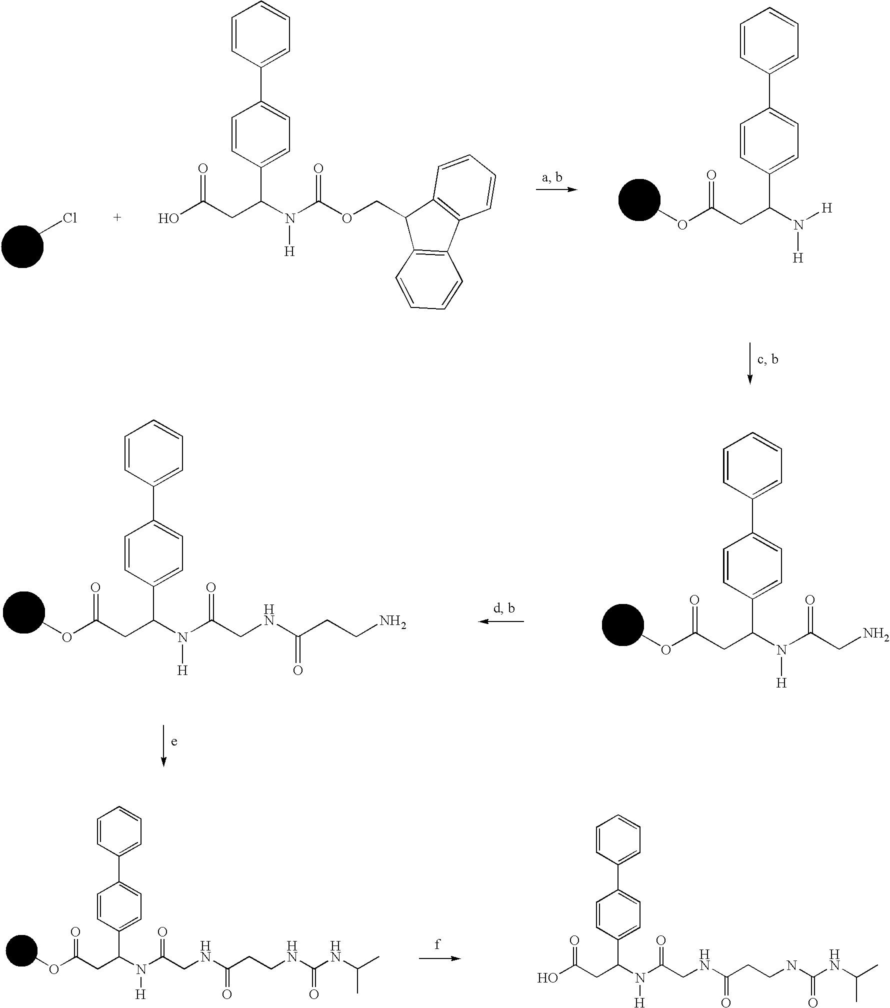

a 7.36 g of trityl resin (Rapp) are suspended with 50 ml of dichloro-methane, and 3.6 ml of diisopropylethylamine are subsequently added. A solution of 6.50 g of Fmoc-diphenylaminopropionic acid in dichoromethane are added to this suspension, and the mixture is subsequently shaken for 4 hours at RT. For work-up, the solid phase is filtered off and washed 3 times with each of dichloromethane, DMF, dichloromethane and methanol and dried in a vacuum drying cabinet.

b The solid phase is suspended with DMF, a 50% solution of piperidine in DMF is subsequently added, and the mixture is shaken for 30 minutes at RT. The solid phase is subsequently filtered off, and the same procedure is repeated twice. The solid phase is subsequently washed three times with each of DMF, dichloromethane and methanol and dried overnight in a vacuum drying cabinet, giving resin-bound 3-biphenyl4-yl-3-aminopropionic acid “AB”.

c 14.098 g of solid phase are suspended in 80 ml of DMF, and 13.83 ml of diisopropylethylamine are added. 21.28 g of Fmoc-glycine, 14.00 g of HOBt and 13.84 ml of diisopropylcarbodiimide as a solution in 130 ml of DMF are subsequently added, and the reaction batch is shaken overnight at RT. For work-up, the solid phase is filtered off, washed three times with each of DMF, dichloromethane and methanol and dried overnight in a vacuum drying cabinet, giving resin-bound 3-biphenyl-4-yl-3-(2-aminoethanoylamino)propionic acid “BC”.

d 5.25 g of polymer are suspended with 30 ml DMF, 5.33 ml of diisopropylethylamine are added, and a solution of 5.90 g of Fmoc-β-alanine, 5.4 g of HOBt and 5.36 ml of diisopropylcarbodiimide is added. This suspension is shaken overnight at RT. 622 μl of diethyl azadicarboxylate are added dropwise. The suspension is stirred over-night at RT. For work-up, the solid phase is filtered off, washed three thimes with each of DMF, dichloromethane and methanol, and dried overnight in a vacuum drying cabinet, giving resin-bound 3-biphenyl-4-yl-3-[2-(3-aminopropanoylamino)ethanoylamino]propionic acid “CD”.

e 150 mg of polymer are suspended in 2 ml dichloromethane, and 187 μl of diisopropylethylamine are added. A solution of 93 mg of isopropyl isocyanate in dichloromethane is added to this suspension, and the reaction batch is shaken overnight at RT. For work-up, the solid phase is filtered off, washed 3 times with each of DMF, dichloro-methane and methanol and dried overnight in a vacuum drying cabinet, giving 3-biphenyl-4-yl-3-{2-[3-(3-isopropylureido)-propanoylamino]ethanolylamino}propionic acid “DE”.

f 164 mg of the polymer are suspended in 1 ml of dichloromethane, 3 ml of a 50% solution of TFA in dichloromethane are added, and the mixture is shaken for 1 hour at RT. The solid phase is removed by filtration, and the solution is evaporated to dryness in a Speedvac, giving 34 mg of the desired product (3-biphenyl-4-yl-3-{2-[3-(3-iso-propylureido)propanoylamino]ethanolylamino}propionic acid; (EMD 388100)) as a slightly brownish oil.

### EXAMPLE 2

Analogously to Example 1, the resin “BC” is reacted with FMOC-protected β-alanine and benzyl isothiocyanate, giving 3-biphenyl-4-yl-3-{2-[3-(3-benzylthioureido)propanoylamino]ethanoylamino}propionic acid. Preparative HPLC gives 3-biphenyl-4-yl-3-{2-[3-(3-benzylthioureido)-propanoylamino]ethanoylamino}propionic acid trifluoroacetate, RT 1.887 min, FAB-MS (M+H)⁺519.15 (EMD 388118).

Analogously to Example 1, the resin “BC” is reacted with FMOC-protected 4-aminobutanoic acid and benzyl isocyanate, giving 3-biphenyl-4-yl-3-{2-[4-(3-benzylureido)butanoylamino]ethanoylamino}propionic acid.

Preparative HPLC gives 3-biphenyl-4-yl-3-{2-[4-(3-benzylureido)butanoyl-amino]ethanoylamino}propionic acid trifluoroacetate, RT 1.627 min, FAB-MS (M+H)⁺517.2 (EMD 387143).

Analogously to Example 1, the resin “BC” is reacted with FMOC-protected 4-aminobutanoic acid and isocyanic acid, giving 3-biphenyl-4-yl-3-{2-[4-ureidobutanoylamino]ethanoylamino}propionic acid.

Preparative HPLC gives 3-biphenyl-4-yl-3-[2-(4-ureidobutanoylamino)-ethanoylamino}propionic acid trifluoroacetate, RT 1.232 min, FAB-MS (M+H)⁺427.1 (EMD 387505).

Analogously to Example 1, the resin “BC” is reacted with FMOC-protected 4-aminobutanoic acid and ethyl isocyanate, giving 3-biphenyl-4-yl-3-{2-[4-(3-ethylureido)butanoylamino]ethanoylamino}propionic acid.

Preparative HPLC gives 3-biphenyl-4-yl-3-{2-[4-(3-ethylureido)butanoylamino]ethanoylamino}propionic acid trifluoroacetate, RT 1.363 min, FAB-MS (M+H)⁺455.2 (EMD 387506).

Analogously to Example 1, the resin “BC” is reacted with FMOC-protected 4-aminobutanoic acid and cyclohexyl isocyanate, giving 3-biphenyl4-yl-3-{2-[4-(3-cyclohexylureido)butanoylamino]ethanoylamino}propionic acid.

Preparative HPLC gives 3-biphenyl-4-yl-3-{2-[4-(3-cyclohexyl-ureido)butanoylamino]ethanoylamino}propionic acid trifluoroacetate, RT 1.688 min, FAB-MS (M+H)⁺508.2 (EMD 387507).

Analogously to Example 1, the resin “BC” is reacted with FMOC-protected 4-aminobutanoic acid and isopropyl isocyanate, giving 3-biphenyl4-yl-3-{2-[4-(3-isoproylureido)butanoylamino]ethanoylamino}propionic acid.

Preparative HPLC gives 3-biphenyl-4-y-3-{2-[4-(3-isoproylureido)butanoyl-amino]ethanoylamino}propionic acid trifluoroacetate, RT 1.454 min, FAB-MS (M+H)⁺462.2 (EMD 387508).

Analogously to Example 1, the resin “BC” is reacted with FMOC-protected 4-aminobutanoic acid and n-butyl isocyanate, giving 3-biphenyl-4-yl-3-{2-[4-(3-butylureido)butanoylamino]ethanoylamino}propionic acid.

Preparative HPLC gives 3-biphenyl4-yl-3-{2-[4-(3-butylureido)butanoyl-amino]ethanoylamino}propionic acid trifluoroacetate, RT 1.588 min, FAB-MS (M+H)⁺483.2 (EMD 387509).

Analogously to Example 1, the resin “BC” is reacted with FMOC-protected 4-aminobutanoic acid and tert-butyl isocyanate, giving 3-biphenyl-4-yl-3-{2-[4-(3-tert-butylureido)butanoylamino]ethanoylamino}propionic acid.

Preparative HPLC gives 3-biphenyl-4-yl-3-{2-[4-(3-tert-butylureido)butanoyl-amino]ethanoylamino}propionic acid trifluoroacetate, RT 1.6 min, FAB-MS (M+H)⁺483.2 (EMD 387510).

Analogously to Example 1, the resin “BC” is reacted with FMOC-protected 4-aminobutanoic acid and methyl isothiocyanat, giving 3-biphenyl4-yl-3-{2-[4-(3-methylthioureido)butanoylamino]ethanoylamino}propionic acid.

Preparative HPLC gives 3-biphenyl-4-yl-3-{2-[4-(3-methylthioureido)-butanoylamino]ethanoylamino}propionic acid trifluoroacetate, RT 1.399 min, FAB-MS (M+H)⁺457.2 (EMD 387511).

Analogously to Example 1, the resin “BC” is reacted with FMOC-protected 4-aminobutanoic acid and methyl isothiocyanat, giving 3-biphenyl-4-yl-3-{2-[4-(3-methylthioureido)butanoylamino]ethanoylamino}propionic acid.

Preparative HPLC gives 3-biphenyl-4-yl-3-{2-[4-(3-methylthioureido)-butanoylamino]ethanoylamino}propionic acid trifluoroacetate, RT 1.399 min, FAB-MS (M+H)⁺457.2 (EMD 387511).

Analogously to Example 1, the resin “BC” is reacted with FMOC-protected 4-aminobutanoic acid and phenyl isocyanate, giving 3-biphenyl-4-yl-3-{2-[4-(3-phenylureido)butanoylamino]ethanoylamino}propionic acid.

Preparative HPLC gives 3-biphenyl-4-yl-3-{2-[4-(3-phenylureido)butanoyl-amino]ethanoylamino}propionic acid trifluoroacetate, RT 1.66 min, FAB-MS (M+H)⁺503.2 (EMD 387512).

Analogously to Example 1, the resin “BC” is reacted with FMOC-protected 4-aminobutanoic acid and phenylethyl isocyanate, giving 3-biphenyl-4-yl-3-{2-[4-(3-phenylethylureido)butanoylamino]ethanoylamino}propionic acid.

Preparative HPLC gives 3-biphenyl4-yl-3-{2-[4-(3-phenylethylureido)-butanoylamino)ethanoylamino}propionic acid trifluoroacetate, RT 1.717 min, FAB-MS (M+H)⁺531.2 (EMD 387513).

Analogously to Example 1, the resin “BC” is reacted with FMOC-protected 4-aminobutanoic acid and 2-chlorophenyl isocyanate, giving 3-biphenyl-4-yl-3-{2-[4-(3-(2-chlorophenyl)ureido)butanoylamino]ethanoylamino}propionic acid.

Preparative HPLC gives 3-biphenyl-4-yl-3-{2-[4-(3-(2-chlorophenyl)ureido)-butanoylamino]ethanoylamino}propionic acid trifluoroacetate, RT 1.778 min, FAB-MS (M+H)⁺537.2/539 (EMD 387514).

Analogously to Example 1, the resin “BC” is reacted with FMOC-protected 4-aminobutanoic acid and 3-chlorophenyl isocyanate, giving 3-biphenyl-4-yl-3-{2-[4-(3-(3-chlorophenyl)ureido)butanoylamino]ethanoylamino}propionic acid.

Preparative HPLC gives 3-biphenyl-4-yl-3-{2-[4-(3-(3-chlorophenyl)-ureido)butanoylamino]ethanoylamino}propionic acid trifluoroacetate, RT 1.883 min, FAB-MS (M+H)⁺537.2/539 (EMD 387515).

Analogously to Example 1, the resin “BC” is reacted with FMOC-protected 4-aminobutanoic acid and 4-chlorophenyl isocyanate, giving 3-biphenyl-4-yl-3-{2-[4-(3-(4-chlorophenyl)ureido)butanoylamino]ethanoylamino}-propionic acid.

Preparative H PLC gives 3-biphenyl-4-yl-3-{2-[4-(3-(4-chlorophenyl)ureido)-butanoylamino]ethanoylamino}propionic acid trifluoroacetate, RT 1.834 min, FAB-MS (M+H)⁺537.2/539 (EMD 387516).

Analogously to Example 1, the resin “BC” is reacted with FMOC-protected 4-aminobutanoic acid and 2-methoxyphenyl isocyanate, giving 3-biphenyl-4-yl-3-{2-[4-(3-(2-methoxyphenyl)ureido)butanoylamino]ethanoylamino}-propionic acid.

Preparative HPLC gives 3-biphenyl-4-yl-3-{2-[4-(3-(2-methoxyphenyl)-ureido)butanoylamino]ethanoylamino}propionic acid trifluoroacetate, RT 1.709 min, FAB-MS (M+H)⁺533.2 (EMD 387517).

Analogously to Example 1, the resin “BC” is reacted with FMOC-protected 4-aminobutanoic acid and 4-methoxyphenyl isocyanate, giving 3-biphenyl-4-yl-3-{2-[4-(3-(4-methoxyphenyl)ureido)butanoylamino]ethanoylamino}-propionic acid.

Preparative HPLC gives 3-biphenyl4-yl-3-{2-[4-(3-(4-methoxyphenyl)-ureido)butanoylamino]ethanoylamino}propionic acid trifluoroacetate, RT 1.622 min, FAB-MS (M+H)⁺533.2 (EMD 387518).

Analogously to Example 1, the resin “BC” is reacted with FMOC-protected β-alanine and isocyanic acid, giving 3-biphenyl-4-yl-3-[2-(3-ureido-propanoylamino)ethanoylamino]propionic acid.

Preparative HPLC gives 3-biphenyl4-yl-3-[2-(3-ureidopropanoylamino)-ethanoylamino]propionic acid trifluoroacetate, RT 1.197 min, FAB-MS (M+H)⁺413.2 (EMD 388097).

Analogously to Example 1, the resin “BC” is reacted with FMOC-protected β-alanine and ethyl isocyanate, giving 3-biphenyl-4-yl-3-{2-[3-(3-ethyl-ureido)propanoylamino]ethanoylamino}propionic acid.

Preparative HPLC gives 3-biphenyl-4-yl-3-{2-[3-(3-ethylureido)propanoylamino]ethanoylamino}propionic acid trifluoroacetate, RT 1.32 min, FAB-MS (M+H)⁺441.2 (EMD 388098).

Analogously to Example 1, the resin “BC” is reacted with FMOC-protected β-alanine and cyclohexyl isocyanate, giving 3-biphenyl-4-yl-3-{2-[3-(3-cyclohexylureido)propanoylamino]ethanoylamino}propionic acid.

Preparative HPLC gives 3-biphenyl-4-yl-3-{2-[3-(3-cyclohexylureido)-propanoylamino]ethanoylamino}propionic acid trifluoroacetate, RT 1.634 min, FAB-MS (M+H)⁺495.2 (EMD 388099).

Analogously to Example 1, the resin “BC” is reacted with FMOC-protected β-alanine and n-butyl isocyanate, giving 3-biphenyl-4-yl-3-{2-[3-(3-butyl-ureido)propanoylamino]ethanoylamino}propionic acid.

Preparative HPLC gives 3-biphenyl-4-yl-3-{2-[3-(3-butylureido)propanoylamino]ethanoylamino}propionic acid trifluoroacetate, RT 1.536 min, FAB-MS (M+H)⁺469.2 (EMD 388101).

Analogously to Example 1, the resin “BC” is reacted with FMOC-protected β-alanine and tert-butyl isocyanate, giving 3-biphenyl-4-yl-3-{2-[3-(3-tert-butylureido)propanoylamino]ethanoylamino}propionic acid.

Preparative HPLC gives 3-biphenyl-4-yl-3-{2-[3-(3-tert-butylureido)-propanoylamino]ethanoylamino}propionic acid trifluoroacetate, RT 1.543 min, FAB-MS (M+H)⁺469.2 (EMD 388102).

Analogously to Example 1, the resin “BC” is reacted with FMOC-protected β-alanine and methyl isothiocyanate, giving 3-biphenyl-4-yl-3-{2-[3-(3-methylthioureido)propanoylamino]ethanoylamino}propionic acid.

Preparative HPLC gives 3-biphenyl-4-yl-3-{2-[3-(3-methylthioureido)-propanoylamino]ethanoylamino}propionic acid trifluoroacetate, RT 1.363 min, FAB-MS (M+H)⁺443.2 (EMD 388103).

Analogously to Example 1, the resin “BC” is reacted with FMOC-protected β-alanine and phenyl isocyanate, giving 3-biphenyl-4-yl-3-{2-[3-(3-phenyl-ureido)propanoylamino]ethanoylamino}propionic acid.

Preparative HPLC gives 3-biphenyl-4-yl-3-{2-[3-(3-phenylureido)propanoyl-amino]ethanoylamino}propionic acid trifluoroacetate, RT 1.615 min, FAB-MS (M+H)⁺489;2 (EMD 388104).

Analogously to Example 1, the resin “BC” is reacted with FMOC-protected β-alanine and phenylethyl isocyanate, giving 3-biphenyl-4-yl-3-{2-[3-(3-phenylethylureido)propanoylamino]ethanoylamino}propionic acid.

Preparative HPLC gives 3-biphenyl-4-yl-3-{2-[3-(3-phenylethylureido)-propanoylamino]ethanoylamino}propionic acid trifluoroacetate, RT 1.662 min, FAB-MS (M+H)⁺517.2 (EMD 388105).

Analogously to Example 1, the resin “BC” is reacted with FMOC-protected β-alanine and 2-chlorophenyl isocyanate, giving 3-biphenyl-4-yl-3-{2-[3-(3-(2-chlorophenylethyl)ureido)propanoylamino]ethanoylamino}propionic acid .

Preparative HPLC gives 3-biphenyl-4-yl-3-{2-[3-(3-(2-chlorophenylethyl)-ureido)propanoylamino]ethanoylamino}propionic acid trifluoroacetate, RT 1.772 min, FAB-MS (M+H)⁺523.2/525 (EMD 388106).

Analogously to Example 1, the resin “BC” is reacted with FMOC-protected β-alanine and 3-chlorophenyl isocyanate, giving 3-biphenyl-4-yl-3-{2-[3-(3-(3-chlorophenylethyl)ureido)propanoylamino]ethanoylamino}propionic acid.

Preparative HPLC gives 3-biphenyl-4-yl-3-{2-[3-(3-(3-chlorophenylethyl)-ureido)propanoylamino]ethanoylamino}propionic acid trifluoroacetate, RT 1.782 min, FAB-MS (M+H)⁺523.2/525 (EMD 388107).

Analogously to Example 1, the resin “BC” is reacted with FMOC-protected β-alanine and 4-chlorophenyl isocyanate, giving 3-biphenyl-4-yl-3-{2-[3-(3-(4-chlorophenylethyl)ureido)propanoylamino]ethanoylamino}propionic acid.

Preparative HPLC gives 3-biphenyl-4-yl-3-{2-[3-(3-(4-chlorophenylethyl)-ureido)propanoylamino]ethanoylamino}propionic acid trifluoroacetate, RT 1.779 min, FAB-MS (M+H)⁺523.2/525 (EMD 388108).

Analogously to Example 1, the resin “BC” is reacted with FMOC-protected β-alanine and 2-methoxyphenyl isocyanate, giving 3-biphenyl-4-yl-3-{2-[3-(3-(2-methoxyphenyl)ureido)propanoylamino]ethanoylamino}propionic acid.

Preparative HPLC gives 3-biphenyl-4-yl-3-{2-[3-(3-(2-methoxyphenyl)-ureido)propanoylamino]ethanoylamino}propionic acid trifluoroacetate, RT 1.65 min, FAB-MS (M+H)⁺519.2 (EMD 388109).

Analogously to Example 1, the resin “BC” is reacted with FMOC-protected β-alanine and 4-methoxyphenyl isocyanate, giving 3-biphenyl-4-yl-3-{2-[3-(3-(4-methoxyphenyl)ureido)propanoylamino]ethanoylamino}propionic acid.

Preparative HPLC gives 3-biphenyl-4-yl-3-{2-[3-(3-(4-methoxyphenyl)-ureido)propanoylamino]ethanoylamino}propionic acid trifluoroacetate, RT 1.572 min, FAB-MS (M+H)⁺519.2 (EMD 388110).

Analogously to Example 1, the resin “BC” is reacted with FMOC-protected β-alanine and 3-methoxyphenyl isocyanate, giving 3-biphenyl-4-yl-3-{2-[3-(3-(3-methoxyphenyl)ureido)propanoylamino]ethanoylamino}propionic acid.

Preparative HPLC gives 3-biphenyl-4-yl-3-{2-[3-(3-(3-methoxyphenyl)-ureido)propanoylamino]ethanoylamino}propionic acid trifluoroacetate, RT 1.624 min, FAB-MS (M+H)⁺519.2 (EMD 388111).

Analogously to Example 1, the resin “BC” is reacted with FMOC-protected β-alanine and (R)-1-phenylethyl isocyanate, giving 3-biphenyl-4-yl-3-{2-[3-(3-((R)-1-phenylethyl)ureido)propanoylamino]ethanoylamino}propionic acid.

Preparative HPLC gives 3-biphenyl-4-yl-3-{2-[3-(3-((R)-1-phenylethyl)-ureido)propanoylamino]ethanoylamino}propionic acid trifluoroacetate, RT 1.653 min, FAB-MS (M+H)⁺517.2 (EMD 388112).

Analogously to Example 1, the resin “BC” is reacted with FMOC-protected β-alanine and (S)-1-phenylethyl isocyanate, giving 3-biphenyl-4-yl-3-{2-[3-(3-((S)-1-phenylethyl)ureido)propanoylamino]ethanoylamino}propionic acid.

Preparative HPLC gives 3-biphenyl-4-yl-3-{2-[3-(3-((S) 1-phenylethyl)-ureido)propanoylamino]ethanoylamino}propionic acid trifluoroacetate, RT 1.656 min, FAB-MS (M+H)⁺517.2 (EMD 388113).

Analogously to Example 1, the resin “BC” is reacted with FMOC-protected β-alanine and (R)-1-naphthalen-1-ylethyl isocyanate, giving 3-biphenyl-4-yl-3-{2-[3-(3-((R)-1-naphthalen-1-ylethyl)ureido)propanoylamino]ethanoyl-amino}propionic acid.

Preparative HPLC gives 3-biphenyl-4-yl-3-{2-[3-(3-((R)-1-naphthalen-1-yl-ethyl)ureido)propanoylamino]ethanoylamino}propionic acid trifluoroacetate, RT 1.854 min, FAB-MS (M+H)⁺567.2 (EMD 388114).

Analogously to Example 1, the resin “BC” is reacted with FMOC-protected β-alanine and (S)-1-naphthalen-1-ylethyl isocyanate, giving 3-biphenyl-4-yl-3-{2-[3-(3-((S)-1-naphthalen-1-ylethyl)ureido)propanoylamino]ethanoyl-amino}propionic acid.

Preparative HPLC gives 3-biphenyl-4-yl-3-{2-[3-(3-((S)-1-naphthalen-1-yl-ethyl)ureido)propanoylamino]ethanoylamino}propionic acid trifluoroacetate, RT 1.851 min, FAB-MS (M+H)⁺567.2 (EMD 388115).

Analogously to Example 1, the resin “BC” is reacted with FMOC-protected β-alanine and naphthalen-1-yl isocyanate, giving 3-biphenyl-4-yl-3-{2-[3-(3-naphthalen-1-ylureido)propanoylamino]ethanoylamino}propionic acid.

Preparative HPLC gives 3-biphenyl4-yl-3-{2-[3-(3-naphthalen-1-ylureido)-propanoylamino]ethanoylamino}propionic acid trifluoroacetate, RT 1.742 min FAB-MS (M+H)⁺539.2 (EMD 388116).

Analogously to Example 1, the resin “BC” is reacted with FMOC-protected β-alanine and naphthalen-2-yl-isocyanate, giving 3-biphenyl-4-yl-342-[3-(3-naphthalen-2-ylureido)propanoylamino]ethanoylamino}propionic acid.

Preparative HPLC gives 3-biphenyl-4-yl-3-{2-[3-(3-naphthalen-2-ylureido)-propanoylamino]ethanoylamino}propionic acid trifluoroacetate, RT 1.829 min, FAB-MS (M+H)⁺539.2 (EMD 388117).

Analogously to Example 1, the resin “BC” is reacted with FMOC-protected β-alanine and benzyl isothiocyanate, giving 3-biphenyl-4-yl-3-{2-[3-(3-benzylthioureido)propanoylamino]ethanoylamino}propionic acid.

Preparative HPLC gives 3-biphenyl-4-yl-3-{2-[3-(3-benzylthioureido)-propanoylamino]ethanoylamino}propionic acid trifluoroacetate, RT 1.77 min, FAB-MS (M+H)⁺519.2 (EMD 388118).

Analogously to Example 1, the resin “BC” is reacted with FMOC-protected β-alanine and 4-methylbenzyl isocyanate, giving 3-biphenyl-4-yl-3-{2-[3-(3-(4-methylbenzyl)ureido)propanoylamino]ethanoylamino}propionic acid.

Preparative HPLC gives 3-biphenyl-4-yl-3-{2-[3-(3-(4-methylbenzyl)ureido)-propanoylamino]ethanoylamino}propionic acid trifluoroacetate, RT 1.696 min, FAB-MS (M+H)⁺517.2 (EMD 388119).

Analogously to Example 1, the resin “BC” is reacted with FMOC-protected β-alanine and 2.4-dichlorobenzyl isocyanate, giving 3-biphenyl-4-yl-3-{2-[3-(3-(2.4-dichlorobenzyl)ureido)propanoylamino]ethanoylamino}propionic acid.

Preparative HPLC gives 3-biphenyl-4-yl-3-{2-[3-(3-(2.4-dichlorobenzyl)-ureido)propanoylamino]ethanoylamino}propionic acid trifluoroacetate, RT 1.852 min, FAB-MS (M+H)⁺572.1 (EMD 388120).

Analogously to Example 1, the resin “BC” is reacted with FMOC-protected β-alanine and 4-fluorobenzyl isocyanate, giving 3-biphenyl-4-yl-3-{2-[3-(3-(4-fluorobenzyl)ureido)propanoylamino]ethanoylamino}propionic acid.

Preparative HPLC gives 3-biphenyl-4-yl-3-{2-[3-(3-(4-fluorobenzyl)ureido)-propanoylamino]ethanoylamino}propionic acid trifluoroacetate, RT 1.632 min, FAB-MS (M+H)⁺521.2 (EMD 388121).

Analogously to Example 1, the resin “BC” is reacted with FMOC-protected β-alanine and 3.4-dichlorobenzyl isocyanate, giving 3-biphenyl-4-yl-3-{2-[3-(3-(3.4-dichlorobenzyl)ureido)propanoylamino]ethanoylamino}propionic acid.

Preparative HPLC gives 3-biphenyl-4-yl-3-{2-[3-(3-(3.4-dichlorobenzyl)-ureido)propanoylamino]ethanoylamino}propionic acid trifluoroacetate, RT 1.833 min, FAB-MS (M+H)⁺572.2 (EMD 388122).

Analogously to Example 1, the resin “BC” is reacted with FMOC-protected β-alanine and 2-chlorobenzyl isocyanate, giving 3-biphenyl-4-yl-3-{2-[3-(3-(2-chlorobenzyl)ureido)propanoylamino]ethanoylamino}propionic acid.

Preparative HPLC gives 3-biphenyl-4-yl-3-{2-[3-(3-(2-chlorobenzyl)ureido)-propanoylamino]ethanoylamino}propionic acid trifluoroacetate, RT 1.694 min, FAB-MS (M+H)⁺537.2/539 (EMD 388123).

Analogously to Example 1, the resin “BC” is reacted with FMOC-protected β-alanine and n-propyl isocyanate, giving 3-biphenyl-4-yl-3-{2-[3-(3-(3-propyl)ureido)propanoylamino]ethanoylamino}propionic acid.

Preparative HPLC gives 3-biphenyl-4-yl-3-{2-[3-(3-(3-propyl)ureido)-propanoylamino]ethanoylamino}propionic acid trifluoroacetate, RT 1.418 min, FAB-MS (M+H)⁺455.2 (EMD 388124).

Analogously to Example 1, the resin “BC” is reacted with FMOC-protected β-alanine and allyl isocyanate, giving 3-biphenyl-4-yl-3-{2-[3-(3-allylureido)-propanoylamino]ethanoylamino}propionic acid.

Preparative HPLC gives 3-biphenyl4-yl-3-{2-[3-(3-allylureido)propanoyl-amino]ethanoylamino}propionic acid trifluoroacetate, RT 1.376 min, FAB-MS (M+H)⁺453.2 (EMD 388125).

Analogously to Example 1, the resin “BC” is reacted with FMOC-protected 5-aminopentanoic acid and isocyanic acid, giving 3-biphenyl-4-yl-3-[2-(5-ureidopentanoylamino)ethanoylamino]propionic acid.

Preparative HPLC gives 3-biphenyl-4-yl-3-[2-(5-ureidopentanoylamino)-ethanoylamino]propionic acid trifluoroacetate, RT 1.225 min, FAB-MS (M+H)⁺441.2 (EMD 388126).

Analogously to Example 1, the resin “BC” is reacted with FMOC-protected 5-aminopentanoic acid and ethylisocyanic acid, giving 3-biphenyl-4-yl-3-{2-[5-(3-ethylureido)pentanoylamino]ethanoylamino}propionic acid.

Preparative HPLC goes 3-biphenyl-4-yl-3-{2-[5-(3-ethylureido)-pentanoylamino]ethanoylamino}propionic acid trifluoroacetate, RT 1.354 min, FAB-MS (M+H)⁺496.2 (EMD 388127).

Analogously to Example 1, the resin “BC” is reacted with FMOC-protected 5-aminopentanoic acid and cyclohexyl isocyanate, giving 3-biphenyl-4-yl-3-{2-[5-(3-cyclohexylureido)pentanoylamino]ethanoylamino}propionic acid.

Preparative HPLC gives 3-biphenyl-4-yl-3-{2-[5-(3-cyclohexylureido)-pentanoylamino]ethanoylamino}propionic acid trifluoroacetate, RT 1.678 min, FAB-MS (M+H)⁺523.2 (EMD 388128).

Analogously to Example 1, the resin “BC” is reacted with FMOC-protected 5-aminopentanoic acid and isopropyl isocyanate, giving 3-biphenyl-4-yl-3-{2-[5-(3-isopropylureido)pentanoylamino]ethanoylamino}propionic acid.

Preparative HPLC gives 3-biphenyl-4-yl-3-{2-[5-(3-isopropylureido)-pentanoylamino]ethanoylamino}propionic acid trifluoroacetate, RT 1.447 min, FAB-MS (M+H)⁺483.2 (EMD 388129).

Analogously to Example 1, the resin “BC” is reacted with FMOC-protected 5-aminopentanoic acid and n-butyl isocyanate, giving 3-biphenyl-4-yl-3-{2-[5-(3-butylureido)pentanoylamino]ethanoylamino}propionic acid.

Preparative HP LC gives 3-biphenyl-4-yl-3-{2-[5-(3-butylureido)-pentanoylamino]ethanoylamino}propionic acid trifluoroacetate, RT 1.572 min, FAB-MS (M+H)⁺497.2 (EMD 388130).

Analogously to Example 1, the resin “BC” is reacted with FMOC-protected 5-aminopentanoic acid and tert-butyl isocyanate, giving 3-biphenyl-4-yl-3-{2-[5-(3-tert-butylureido)pentanoylamino]ethanoylamino}propionic acid.

Preparative HPLC gives 3-biphenyl-4-yl-3-{2-[5-(3-tert-butylureido)-pentanoylamino)ethanoylamino}propionic acid trifluoroacetate, RT 1.588 min, FAB-MS (M+H)⁺497.2 (EMD 388131).

Analogously to Example 1, the resin “BC” is reacted with FMOC-protected 5-aminopentanoic acid and Methylisothiocyanat, giving 3-biphenyl-4-yl-3-{2-[5-(3-methylthioureido)pentanoylamino]ethanoylamino}propionic acid.

Preparative HPLC gives 3-biphenyl-4-yl-3-{2-[5-(3-methylthioureido)-pentanoylamino]ethanoylamino}propionic acid trifluoroacetate, RT 1.393 min, FAB-MS (M+H)⁺471.2 (EMD 388132).

Analogously to Example 1, the resin “BC” is reacted with FMOC-protected 5-aminopentanoic acid and phenyl isocyanate, giving 3-biphenyl-4-yl-3-{2-[5-(3-phenylureido)pentanoylamino]ethanoylamino}propionic acid.

Preparative HPLC gives 3-biphenyl-4-yl-3-{2-[5-(3-phenylureido)-pentanoylamino]ethanoylamino}propionic acid trifluoroacetate, RT 1.645 min, FAB-MS (M+H)⁺517.2 (EMD 388133).

Analogously to Example 1, the resin “BC” is reacted with FMOC-protected 5-aminopentanoic acid and phenylethyl isocyanate, giving 3-biphenyl-4-yl-3-{2-[5-(3-phenylethylureido)pentanoylamino]ethanoylamino}propionic acid.

Preparative HPLC gives 3-biphenyl-4-yl-3-{2-[5-(3-phenylethylureido)-pentanoylamino]ethanoylamino}propionic acid trifluoroacetate, RT 1.698 min, FAB-MS (M+H)⁺545.2 (EMD 388134).

Analogously to Example 1, the resin “BC” is reacted with FMOC-protected 5-aminopentanoic acid and 2-chlorophenyl isocyanate, giving 3-biphenyl-4-yl-3-{2-[5-(3-(2-chlorophenyl)ureido)pentanoylamino]ethanoylamino}-propionic acid.

Preparative HPLC gives 3-biphenyl-4-yl-3-{2-[5-(3-(2-chlorophenyl)ureido)-pentanoylamino]ethanoylamino}propionic acid trifluoroacetate, RT 1.768 min, FAB-MS (M+H)⁺551.2/553 (EMD 388135).

Analogously to Example 1, the resin “BC” is reacted with FMOC-protected 5-aminopentanoic acid and 3-chlorophenyl isocyanate, giving 3-biphenyl-4-yl-3-{2-[5-(3-(3-chlorophenyl) ureido)pentanoylamino]ethanoylamino}propionic acid.

Preparative HPLC gives 3-biphenyl-4-yl-3-{2-[5-(3-(3-chlorophenyl)ureido)-pentanoylamino]ethanoylamino}propionic acid trifluoroacetate, RT 1.823 min, FAB-MS (M+H)⁺551.2/553 (EMD 388136).

Analogously to Example 1, the resin “BC” is reacted with FMOC-protected 5-aminopentanoic acid and 4-chlorophenyl isocyanate, giving 3-biphenyl-4-yl-3-{2-[5-(3-(4-chlorophenyl)ureido)pentanoylamino]ethanoylamino}propionic acid.

Preparative HPLC gives 3-biphenyl-4-yl-3-{2-[5-(3-(4-chlorophenyl)ureido)-pentanoylamino]ethanoylamino}propionic acid trifluoroacetate, RT 1.816 min, FAB-MS (M+H)⁺551,04/553 (EMD 388137).

Analogously to Example 1, the resin “BC” is reacted with FMOC-protected 5-aminopentanoic acid and 2-methoxyphenyl isocyanate, giving 3-biphenyl-4-yl-3-{2-[5-(3-(2-methoxyphenyl)ureido)pentanoylamino]ethanoylamino}-propionic acid.

Preparative HPLC gives 3-biphenyl-4-yl-3-{2-[5-(3-(2-methoxyphenyl)-ureido)pentanoylamino]ethanoylamino}propionic acid trifluoroacetate, RT 1.693 min, FAB-MS (M+H)⁺547.2 (EMD 388138).

Analogously to Example 1, the resin “BC” is reacted with FMOC-protected 5-aminopentanoic acid and 4-methoxyphenyl isocyanate, giving 3-biphenyl-4-yl-3-{2-[5-(3-(4-methoxyphenyl)ureido)pentanoylamino]ethanoylamino}-propionic acid.

Preparative HPLC gives 3-biphenyl-4-yl-3-{2-[5-(3-(4-methoxyphenyl)-ureido)pentanoylamino]ethanoylamino}propionic acid trifluoroacetate, RT 1.602 min, FAB-MS (M+H)⁺547.2 (EMD 388139).

Analogously to Example 1, the resin “BC” is reacted with FMOC-protected 5-aminopentanoic acid and 3-methoxyphenyl isocyanate, giving 3-biphenyl-4-yl-3-{2-[5-(3-(3-methoxyphenyl)ureido)pentanoylamino]ethanoylamino}-propionic acid.

Preparative HPLC gives 3-biphenyl-4-yl-3-{2-[5-(3-(3-methoxyphenyl)-ureido)pentanoylamino]ethanoylamino}propionic acid trifluoroacetate, RT 1.660 min, FAB-MS (M+H)⁺547.2 (EMD 388140).

Analogously to Example 1, the resin “BC” is reacted with FMOC-protected 3-amino-propansaure and (9H-fluoren-9-yl)methyl formate, giving 3-biphenyl-4-yl-3-{2-[3-(9H-fluoren-9-yl-methoxycarbonylamino)propanoylamino]ethanoylamino}propionic acid.

Preparative HPLC gives 3-biphenyl-4-yl-3-{2-[3-(9H-fluoren-9-yl-methoxycarbonylamino)propanoylamino]ethanoylamino}propionic acid trifluoroacetate, RT 2,089 min, FAB-MS (M+H)⁺529.2 (EMD 388141).

Analogously to Example 1, the resin “BC” is reacted with FMOC-protected 4-aminobutanoic acid and (9H-fluoren-9-yl)methyl formate, giving 3-biphenyl-4-yl-3-{2-[4-(9H-fluoren-9-yl-methoxycarbonylamino)butanoyl-amino]ethanoylamino}propionic acid.

Preparative HPLC gives 3-biphenyl-4-yl-3-{2-[4-(9H-fluoren-9-yl-methoxycarbonylamino)butanoylamino]ethanoylamino}propionic acid trifluoroacetate, RT 2.120 min, FAB-MS (M+H)⁺606.2 (EMD 388142).

Analogously to Example 1, the resin “BC” is reacted with FMOC-protected 5-aminopentanoic acid and (9H-fluoren-9-yl)methyl formate, giving 3-biphenyl-4-yl-3-2-[5-(9H-fluoren-9-yl-methoxycarbonylamino)-pentanoylamino]ethanoylamino}propionic acid.

Preparative HPLC gives 3-biphenyl-4-yl-3-{2-[5-(9H-fluoren-9-yl-methoxycarbonylamino)pentanoylamino]ethanoylamino}propionic acid trifluoroacetate, RT 2.142 min, FAB-MS (M+H)⁺620,2 (EMD 388143).

Analogously to Example 1, the resin “BC” is reacted with FMOC-protected 4-aminobutanoic acid and (R)-1-phenylethyl isocyanate, giving 3-biphenyl-4-yl-3-{2-[4-(3-((R)-1-phenylethyl)ureido)butanoylamino]ethanoylamino}-propionic acid.

Preparative HPLC gives 3-biphenyl-4-yl-3-{2-[4-(3-((R)-1-phenylethyl)-ureido)butanoylamino]ethanoylamino}propionic acid trifluoroacetate, RT 1.677 min, FAB-MS (M+H)⁺531.2 (EMD 388181).

Analogously to Example 1, the resin “BC” is reacted with FMOC-protected 4-aminobutanoic acid and (S)-1-phenylethyl isocyanate, giving 3-biphenyl-4-yl-3-{2-[4-(3-((S)-1-phenylethyl)ureido)butanoylamino]ethanoylamino}propionic acid.

Preparative HPLC gives 3-biphenyl-4-yl-3-{2-[4-(3-((S)-1-phenylethyl)-ureido)butanoylamino]ethanoylamino}propionic acid trifluoroacetate, RT 1.674 min, FAB-MS (M+H)⁺531.2 (EMD 388182).

Analogously to Example 1, the resin “BC” is reacted with FMOC-protected 4-aminobutanoic acid and naphthalen-1-yl isocyanate, giving 3-biphenyl-4-yl-3-{2-[4-(3-naphthalen-1-ylureido)butanoylamino]ethanoylamino}propionic acid.

Preparative H PLC gives 3-biphenyl4-yl-3-{2-[4-(3-naphthalen-1-yl-ureido)butanoylamino]ethanoylamino}propionic acid trifluoroacetate, RT 1.756 min, FAB-MS (M+H)⁺553.2 (EMD 388183).

Analogously to Example 1, the resin “BC” is reacted with FMOC-protected 4-aminobutanoic acid and benzyl isothiocyanate, giving 3-biphenyl-4-yl-3-{2-[4-(3-benzylthioureido)butanoylamino]ethanoylamino}propionic acid.

Preparative HPLC gives erhält 3-biphenyl-4-yl-3-{2-[4-(3-benzyl-thioureido)butanoylamino]ethanoylamino}propionic acid trifluoroacetate, RT 1.765 min, FAB-MS (M+H)⁺533.2 (EMD 388185).

Analogously to Example 1, the resin “BC” is reacted with FMOC-protected 4-aminobutanoic acid and naphthalen-2-yl isocyanate, giving 3-biphenyl-4-yl-3-{2-[4-(3-naphthalen-2-ylureido)butanoylamino]ethanoylamino}propionic acid.

Preparative HPLC gives 3-biphenyl-4-yl-3-{2-[4-(3-naphthalen-2-yl-ureido)butanoylamino]ethanoylamino}propionic acid trifluoroacetate, RT 1.842 min, FAB-MS (M+H)⁺553.2 (EMD 388186).

Analogously to Example 1, the resin “BC” is reacted with FMOC-protected 4-aminobutanoic acid and 4-methylbenzyl isocyanate, giving 3-biphenyl-4-yl-3-{2-[4-(3-(4-methylbenzyl)ureido)butanoylamino]ethanoylamino}propionic acid.

Preparative HPLC gives 3-biphenyl-4-yl-3-{2-[4-(3-(4-methylbenzyl)-ureido)butanoylamino]ethanoylamino}propionic acid trifluoroacetate, RT 1.706 min, FAB-MS (M+H)⁺531.2 (EMD 388187).

Analogously to Example 1, the resin “BC” is reacted with FMOC-protected 4-aminobutanoic acid and 3-methylbenzyl isocyanate, giving 3-biphenyl-4-yl-3-{2-[4-(3-(3-methylbenzyl)ureido)butanoylamino]ethanoylamino}propionic acid.

Preparative HPLC gives 3-biphenyl-4-yl-3-{2-[4-(3-(3-methylbenzyl)-ureido)butanoylamino]ethanoylamino}propionic acid trifluoroacetate, RT 1.705 min, FAB-MS (M+H)⁺531.2 (EMD 388188).

Analogously to Example 1, the resin “BC” is reacted with FMOC-protected 4-aminobutanoic acid and 2-Methylbenzyl isocyanate, giving 3-biphenyl-4-yl-3-{2-[4-(3-(2-methylbenzyl)ureido)butanoylamino]ethanoylamino}propionic acid.

Preparative HPLC gives 3-biphenyl-4-yl-3-{2-[4-(3-(2-methylbenzyl)-ureido)butanoylamino]ethanoylamino}propionic acid trifluoroacetate, RT 1.691 min, FAB-MS (M+H)⁺531.2 (EMD 388189).

Analogously to Example 1, the resin “BC” is reacted with FMOC-protected 4-aminobutanoic acid and 2.4-dichlorobenzyl isocyanate, giving 3-biphenyl-4-yl-3-{2-[4-(3-(2.4-dichlorobenzyl)ureido)butanoylamino]ethanoylamino}propionic acid.

Preparative HPLC gives 3-biphenyl-4-yl-3-{2-[4-(3-(2.4-dichlorobenzyl)-ureido)butanoylamino]ethanoylamino}propionic acid trifluoroacetate, RT 1.864 min, FAB-MS (M+H)⁺585.2/587 (EMD 388190).

Analogously to Example 1, the resin “BC” is reacted with FMOC-protected 4-aminobutanoic acid and 4-fluorobenzyl isocyanate, giving 3-biphenyl-4-yl-3-{2-[4-(3-(4-fluorobenzyl)ureido)butanoylamino]ethanoylamino}propionic acid.

Preparative H PLC gives 3-biphenyl-4-yl-3-{2-[4-(3-(4-fluorobenzyl)-ureido)butanoylamino]ethanoylamino}propionic acid trifluoroacetate, RT 1.646 min, FAB-MS (M+H)⁺535.2 (EMD 388191).

Analogously to Example 1, the resin “BC” is reacted with FMOC-protected 4-aminobutanoic acid and 3.4-dichlorobenzyl isocyanate, giving 3-biphenyl-4-yl-3-{2-[4-(3-(3.⁴-dichlorobenzyl)ureido)butanoylamino]ethanoylamino}propionic acid.

Preparative HPLC gives 3-biphenyl-4-yl-3-{2-[4-(3-(3.4-dichlorobenzyl)-ureido)butanoylamino]ethanoylamino}propionic acid trifluoroacetate, RT 1.851 min, FAB-MS (M+H)⁺585.2/587 (EMD 388192).

Analogously to Example 1, the resin “BC” is reacted with FMOC-protected 4-aminobutanoic acid and 2-chlorobenzyl isocyanate, giving 3-biphenyl-4-yl-3-{2-[4-(3-(2-chlorobenzyl)ureido)butanoylamino]ethanoylamino}-propionic acid.

Preparative HPLC gives 3-biphenyl-4-yl-3-{2-[4-(3-(2-chlorobenzyl)-ureido)butanoylamino]ethanoylamino}propionic acid trifluoroacetate, RT 1.713 min, FAB-MS (M+H)⁺551.2/553 (EMD 388193).

Analogously to Example 1, the resin “BC” is reacted with FMOC-protected 4-aminobutanoic acid and 4-methoxybenzyl isocyanate, giving 3-biphenyl-4-yl-3-{2-[4-(3-(4-methoxybenzyl)ureido)butanoylamino]ethanoylamino}-propionic acid.

Preparative HPLC gives erhält 3-biphenyl-4-yl-3-{2-[4-(3-(4-methoxy-benzyl)ureido)butanoylamino]ethanoylamino}propionic acid trifluoroacetate, RT 1.598 min, FAB-MS (M+H)⁺547.2 (EMD 388194).

Analogously to Example 1, the resin “BC” is reacted with FMOC-protected 4-aminobutanoic acid and n-propyl isocyanate, giving 3-biphenyl-4-yl-3-{2-[4-(3-propylureido)butanoylamino]ethanoylamino}propionic acid.

Preparative HPLC gives 3-biphenyl-4-yl-3-{2-[4-(3-propylureido)butanoyl-amino]ethanoylamino}propionic acid trifluoroacetate, RT 1.432 min, FAB-MS (M+H)⁺469.2 (EMD 388195).

Analogously to Example 1, the resin “BC” is reacted with FMOC-protected 4-aminobutanoic acid and allyl isocyanate, giving 3-biphenyl-4-yl-3-{2-[4-(3-allylureido)butanoylamino]ethanoylamino}propionic acid.

Preparative HPLC gives 3-biphenyl-4-yl-3-{2-[4-(3-allylureido)butanoyl-amino]ethanoylamino}propionic acid trifluoroacetate, RT 1.393 min, FAB-MS (M+H)⁺467.2 (EMD 388196).

Analogously to Example 1, the resin “BC” is reacted with FMOC-protected 5-aminopentanoic acid and (R)-1-phenylethyl isocyanate, giving 3-biphenyl-4-yl-3-{2-[5-(3-((R)-1-phenylethyl)ureido)pentanoylamino]ethanoylamino}propionic acid.

Preparative HPLC gives 3-biphenyl-4-yl-3-{2-[5-(3-((R)-1-phenylethyl)-ureido)pentanoylamino]ethanoylamino}propionic acid trifluoroacetate, RT 1.694 min, FAB-MS (M+H)⁺545.2 (EMD 388197).

Analogously to Example 1, the resin “BC” is reacted with FMOC-protected 5-aminopentanoic acid and (S)-1-phenylethyl isocyanate, giving 3-biphenyl-4-yl-3-{2-[5-(3-((S)-1-phenylethyl)ureido)pentanoylamino]ethanoylamino}propionic acid.

Preparative HPLC gives 3-biphenyl-4-yl-3-{2-[5-(3-((S)-1-phenylethyl)-ureido)pentanoylamino]ethanoylamino}propionic acid trifluoroacetate, RT 1.698 min, FAB-MS (M+H)⁺545.2 (EMD 388198).

Analogously to Example 1, the resin “BC” is reacted with FMOC-protected 5-aminopentanoic acid and naphthalen-1-yl isocyanate, giving 3-biphenyl-4-yl-3-{2-[5-(3-naphthalen-1-ylureido)pentanoylamino]ethanoylamino}-propionic acid.

Preparative HPLC gives 3-biphenyl-4-yl-3-{2-[5-(3-naphthalen-1-ylureido)-pentanoylamino]ethanoylamino}propionic acid trifluoroacetate, RT 1.780 min, FAB-MS (M+H)⁺567.2 (EMD 388199).

Analogously to Example 1, the resin “BC” is reacted with FMOC-protected 5-aminopentanoic acid and benzyl isothiocyanate, giving 3-biphenyl-4-yl-3-{2-[5-(3-benzylthioureido)pentanoylamino]ethanoylamino}propionic acid.

Preparative H PLC gives 3-biphenyl-4-yl-3-{2-[5-(3-benzylthioureido)-pentanoylamino]ethanoylamino}propionic acid trifluoroacetate, RT 1.788 min, FAB-MS (M+H)⁺547.3 (EMD 388200).

Analogously to Example 1, the resin “BC” is reacted with FMOC-protected 5-aminopentanoic acid and naphthalen-2-yl isocyanate, giving 3-biphenyl-4-yl-3-{2-[5-(3-naphthalen-2-ylureido)pentanoylamino]ethanoylamino}-propionic acid.

Preparative HPLC gives 3-biphenyl-4-yl-3-{2-[5-(3-naphthalen-2-ylureido)-pentanoylamino]ethanoylamino}propionic acid trifluoroacetate, RT 1.862 min, FAB-MS (M+H)⁺567.2 (EMD 388201).

Analogously to Example 1, the resin “BC” is reacted with FMOC-protected 5-aminopentanoic acid and 4-methylbenzyl isocyanate, giving 3-biphenyl-4-yl-3-{2-[5-(3-(4-methylbenzyl)ureido)pentanoylamino]ethanoylamino}propionic acid.

Preparative HPLC gives 3-biphenyl-4-yl-3-{2-[5-(3-(4-methylbenzyl)ureido)-pentanoylamino]ethanoylamino}propionic acid trifluoroacetate, RT 1.729 min, FAB-MS (M+H)⁺545.2 (EMD 388202).

Analogously to Example 1, the resin “BC” is reacted with FMOC-protected 5-aminopentanoic acid and 3-methylbenzyl isocyanate, giving 3-biphenyl-4-yl-3-{2-[5-(3-(3-methylbenzyl)ureido)pentanoylamino]ethanoylamino}-propionic acid.

Preparative HPLC gives 3-biphenyl-4-yl-3-{2-[5-(3-(3-methylbenzyl)ureido)-pentanoylamino]ethanoylamino}propionic acid trifluoroacetate, RT 1.724 min, FAB-MS (M+H)⁺545.2 (EMD 388203).

Analogously to Example 1, the resin “BC” is reacted with FMOC-protected 5-aminopentanoic acid and 2-methylbenzyl isocyanate, giving 3-biphenyl-4-yl-3-{2-[5-(3-(2-methylbenzyl)ureido)pentanoylamino]ethanoylamino}propionic acid.

Preparative HPLC gives 3-biphenyl-4-yl-3-{2-[5-(3-(2-methylbenzyl)ureido)-pentanoylamino]ethanoylamino}propionic acid trifluoroacetate, RT 1.708 min, FAB-MS (M+H)⁺545.2 (EMD 388204).

Analogously to Example 1, the resin “BC” is reacted with FMOC-protected 5-aminopentanoic acid and 2.4-dichlorobenzyl isocyanate, giving 3-biphenyl-4-yl-3-{2-[5-(3-(2.4-dichlorobenzyl)ureido)pentanoylamino]-ethanoylamino}propionic acid.

Preparative HPLC gives 3-biphenyl-4-yl-3-{2-[5-(3-(2.4-dichlorobenzyl)-ureido)pentanoylamino]ethanoylamino}propionic acid trifluoroacetate, RT 1.891 min, FAB-MS (M+H)⁺600,21 (EMD 388205).

Analogously to Example 1, the resin “BC” is reacted with FMOC-protected 5-aminopentanoic acid and 4-fluorobenzyl isocyanate, giving 3-biphenyl-4-yl-3-{2-(5-(3-(4-fluorobenzyl)ureido)pentanoylamino]ethanoylamino}-propionic acid.

Preparative HPLC gives 3-biphenyl-4-yl-3-{2-[5-(3-(4-fluorobenzyl)ureido)-pentanoylamino]ethanoylamino}propionic acid trifluoroacetate, RT 1.669 min, FAB-MS (M+H)⁺549.2 (EMD 388206).

Analogously to Example 1, the resin “BC” is reacted with FMOC-protected 5-aminopentanoic acid and 3.4-dichlorobenzyl isocyanate, giving 3-biphenyl-4-yl-3-{2-[5-(3-(3.4-dichlorobenzyl)ureido)pentanoylamino]-ethanoylamino}propionic acid.

Preparative HPLC gives 3-biphenyl-4-yl-3-{2-[5-(3-(3.4-dichlorobenzyl)-ureido)pentanoylamino]ethanoylamino}propionic acid trifluoroacetate, RT 1.875 min, FAB-MS (M+H)⁺600,2/(EMD 388207).

Analogously to Example 1, the resin “BC” is reacted with FMOC-protected 5-aminopentanoic acid and 2-chlorobenzyl isocyanate, giving 3-biphenyl-4-yl-3-{2-[5-(3-(2-chlorobenzyl)ureido)pentanoylamino]ethanoylamino}-propionic acid.

Preparative HPLC gives 3-biphenyl-4-yl-3-{2-[5-(3-(2-chlorobenzyl)ureido)-pentanoylamino]ethanoylamino}propionic acid trifluoroacetate, RT 1.735 min, FAB-MS (M+H)⁺565.2/567.2 (EMD 388208).

Analogously to Example 1, the resin “BC” is reacted with FMOC-protected 5-aminopentanoic acid and 4-methoxybenzyl isocyanate, giving 3-biphenyl-4-yl-3-{2-[5-(3-(4-methoxybenzyl)ureido)pentanoylamino]ethanoylamino}propionic acid.

Preparative HPLC gives 3-biphenyl-4-yl-3-{2-[5-(3-(4-methoxybenzyl)-ureido)pentanoylamino]ethanoylamino}propionic acid trifluoroacetate, RT 1.622 min, FAB-MS (M+H)⁺561.2 (EMD 388209).

Analogously to Example 1, the resin “BC” is reacted with FMOC-protected 5-aminopentanoic acid and n-propyl isocyanate, giving 3-biphenyl-4-yl-3-{2-[5-(3-propylureido)pentanoylamino]ethanoylamino}propionic acid.

Preparative HPLC gives 3-biphenyl-4-yl-3-{2-[5-(3-propylureido)-pentanoylamino]ethanoylamino}propionic acid trifluoroacetate, RT 1.451 min, FAB-MS (M+H)⁺483.2 (EMD 388210).

Analogously to Example 1, the resin “BC” is reacted with FMOC-protected 5-aminopentanoic acid and allyl isocyanate, giving 3-biphenyl-4-yl-3-{2-[5-(3-allylureido)pentanoylamino]ethanoylamino}propionic acid.

Preparative HPLC gives 3-biphenyl-4-yl-3-{2-[5-(3-allylureido)pentanoylamino]ethanoylamino}propionic acid trifluoroacetate, RT 1.414 min, FAB-MS (M+H)⁺481.2 (EMD 388211).

Analogously to Example 1, the resin “BC” is reacted with FMOC-protected 3-aminopropanoic acid and ethyl formate, giving 3-biphenyl-4-yl-3-[2-(3-ethoxycarbonylaminopropanoylamino)ethanoylamino]propionic acid.

Preparative HPLC gives 3-biphenyl-4-yl-3-[2-(3-ethoxycarbonylaminopropanoylamino)ethanoylamino]propionic acid trifluoroacetate, RT 1.467 min, FAB-MS (M+H)⁺442.2 (EMD 391898).

Analogously to Example 1, the resin “BC” is reacted with FMOC-protected 3-aminopropanoic acid and benzyl formate, giving 3-biphenyl-4-yl-3-[2-(3-benzyloxycarbonylaminopropanoylamino)ethanoylamino]propionic acid.

Preparative HPLC gives biphenyl-4-yl-3-[2-(3-benzyloxycarbonylaminopropanoylamino)ethanoylamino]propionic acid trifluoroacetate, RT 1.758 min, FAB-MS (M+H)⁺504.2 (EMD 391899).

Analogously to Example 1, the resin “BC” is reacted with FMOC-protected 3-aminopropanoic acid and 2,2-dimethylpropyl formate, giving 3-biphenyl-4-yl-3-{2-[3-(2,2-dimethyl-propoxycarbonylamino)propanoylamino]ethanoylamino}propionic acid.

Preparative HPLC gives 3-biphenyl-4-yl-3-{2-[3-(2,2-dimethyl-propoxy-carbonylamino) propanoylamino]ethanoylamino}propionic acid trifluoro-acetate, RT 1.801 min, FAB-MS (M+H)⁺484.2 (EMD 391900).

Analogously to Example 1, the resin “BC” is reacted with FMOC-protected 4-aminobutanoic acid and ethyl formate, giving 3-biphenyl-4-yl-3-[2-(4-ethoxycarbonylaminobutanoylamino)ethanoylamino]propionic acid.

Preparative HPLC gives 3-biphenyl-4-yl-3-[2-(4-ethoxycarbonylaminobutanoylamino)ethanoylamino]propionic acid trifluoroacetate, RT 1.501 min, FAB-MS (M+H)⁺456.2 (EMD 391901).

Analogously to Example 1, the resin “BC” is reacted with FMOC-protected 4-aminobutanoic acid and benzyl formate, giving 3-biphenyl-4-yl-3-[2-(4-benzyloxycarbonylaminobutanoylamino)ethanoylamino]propionic acid.

Preparative HPLC gives 3-biphenyl-4-yl-3-[2-(4-benzyloxycarbonylamino-butanoylamino)ethanoylamino]propionic acid trifluoroacetate, RT 1.789 min, FAB-MS (M+H)⁺518.2 (EMD 391902).

Analogously to Example 1, the resin “BC” is reacted with FMOC-protected 4-aminobutanoic acid and 2,2-dimethyl-propyl formate, giving 3-biphenyl-4-yl-3-{2-[4-(2,2-dimethyl-propoxycarbonylamino)butanoylamino]ethanoyl-amino}propionic acid.

Preparative HPLC gives 3-biphenyl-4-yl-3-{2-[4-(2,2-dimethyl-propoxy-carbonylamino)butanoylamino]ethanoylamino}propionic acid trifluoro-acetate, RT 1.842 min, FAB-MS (M+H)⁺498.2 (EMD 391903).

The examples below relate to pharmaceutical preparations:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

### EXAMPLE I

### Inhalation Spray

14 g of active ingredient of the formula I are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg.

1. Compounds of the formula I 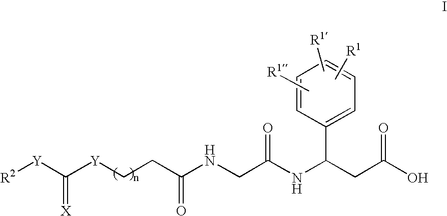
in which
X is O or S
Y independently of one another are NH, O or S
R¹, R^{1′} and R^{1″} are H, A, Ar, Het, Hal, NO₂, CN, OH, OA, NH₂, NHA, NA₂, COOH, COOA, CONH₂, CONHA or CONA₂
R² is H, A, alkenyl having from 1 to 8 carbon atoms and from 1 to 2 double bonds, (CH₂)ₘAr, (CH₂)ₘHet, (CH₂)ₘcycloalkyl, (CH₂)ₘCHAAr, (CH₂)ₘCHAHet or (CH₂)ₘCHA-cycloalkyl,
A is alkyl having from 1 to 8 carbon atoms,
Het is an aromatic monocyclic or bicyclic heterocyclic radical having from 1 to 4 N, O and/or S atoms, which may be unsubstituted or monosubstituted or disubstituted by Hal, A, OH, OA, SA, OCF₃, —CO—A, CN, COOA, COOH, CONH₂, CONHA, CONA₂, NH₂, NHA, NA₂ and/or NO₂,
m is 0, 1 or 2
n is 1,2, 3 or 4
their stereoisomers and their physiologically acceptable salts and solvates.

2. Compounds of the formula I according to claim 1, characterised in that they are
3-biphenyl-4-yl-3-{2-[3-(3-benzylthioureido)propanoylamino]ethanoylamino}propionic acid,
3-biphenyl-4-yl-3{2-[4-(3-benzylureido)butanoylamino]ethanoylamino}-propionic acid,
3-biphenyl-4-yl-3-[2-(4-ureidobutanoylamino)ethanoylamino}propionic acid,
3-biphenyl-4-yl-3-{2-[4-(3-ethylureido)butanoylamino]ethanoylamino}-propionic acid,
3-biphenyl-4-yl-3-{2-[4-(3-cyclohexylureido)butanoylamino]ethanoyl-amino}propionic acid,
3-biphenyl-4-yl-3-{2-[4-(3-isoproylureido)butanoylamino]ethanoylamino}-propionic acid,
3-biphenyl-4-yl-3-{2-[4-(3-butylureido)butanoylamino]ethanoylamino}-propionic acid,
3-biphenyl-4-yl-3-{2-[4-(3-tert-butylureido)butanoylamino]ethanoylamino}propionic acid,
3-biphenyl-4-yl-3-{2-[4-(3-methylthioureido)butanoylamino]ethanoylamino}propionic acid,
3-biphenyl-4-yl-3-{2-[4-(3-methylthioureido)butanoylamino]ethanoylamino}propionic acid,
3-biphenyl-4-yl-3-{2-[4-(3-phenylureido)butanoylamino]ethanoylamino}-propionic acid,
3-biphenyl-4-yl-3-{2-[4-(3-phenylethylureido)butanoylamino]ethanoyl-amino}propionic acid,
3-biphenyl-4-yl-3-{2-[4-(3-(2-chlorophenyl)ureido)butanoylamino]-ethanoylamino}propionic acid,
3-biphenyl-4-yl-3-{2-[4-(3-(3-chlorophenyl)ureido)butanoylamino]-ethanoylamino}propionic acid,
3-biphenyl-4-yl-3-{2-[4-(3-(4-chlorophenyl)ureido)butanoylamino]-ethanoylamino}propionic acid,
3-biphenyl-4-yl-3-{2-[4-(3-(2-methoxyphenyl)ureido)butanoylamino]-ethanoylamino}propionic acid,
3-biphenyl-4-yl-3-{2-(4-(3-(4-methoxyphenyl)ureido)butanoylamino]-ethanoylamino}propionic acid,
3-biphenyl-4-yl-3-[2-(3-ureidopropanoylamino)ethanoylamino]propionic acid,
3-biphenyl-4-yl-3-{2-[3-(3-ethylureido)propanoylamino]ethanoylamino}-propionic acid,
3-biphenyl-4-yl-3-{2-[3-(3-cyclohexylureido)propanoylamino]ethanoylamino}propionic acid,
3-biphenyl-4-yl-3-{2-[3-(3-butylureido)propanoylamino]ethanoylamino}-propionic acid,
3-biphenyl-4-yl-3-{2-[3-(3-tert-butylureido)propanoylamino]ethanoyl-amino}propionic acid,
3-biphenyl-4-yl-3-{2-[3-(3-methylthioureido)propanoylamino]ethanoyl-amino}propionic acid,
3-biphenyl-4-yl-3-{2-[3-(3-phenylureido)propanoylamino]ethanoylamino}-propionic acid,
3-biphenyl-4-yl-3-{2-[3-(3-phenylethylureido) propanoylamino]ethanoylamino}propionic acid,
3-biphenyl-4-yl-3-{2-[3-(3-(2-chlorophenylethyl)ureido)propanoylamino]-ethanoylamino}propionic acid,
3-biphenyl-4-yl-3-{2-[(3-(3-(3-chlorophenylethyl)ureido)propanoylamino]-ethanoylamino}propionic acid,
3-biphenyl-4-yl-3-{2-(3-(3-(4-chlorophenylethyl)ureido)propanoylamino]-ethanoylamino}propionic acid,
3-biphenyl-4-yl-3-{2-[3-(3-(2-methoxyphenyl)ureido)propanoylamino]-ethanoylamino}propionic acid,
3-biphenyl-4-yl-3-{2-[3-(3-(4-methoxyphenyl) ureido)propanoylamino]-ethanoylamino}propionic acid,
3-biphenyl-4-yl-3-{2-[3-(3-(3-methoxyphenyl)ureido)propanoylamino]-ethanoylamino}propionic acid,
3-biphenyl-4-yl-3-{2-[3-(3-((R)-1-phenylethyl)ureido)propanoylamino]-ethanoylamino}propionic acid,
3-biphenyl-4-yl-3-{2-[3-(3-((S)-1-phenylethyl)ureido)propanoylamino]-ethanoylamino}propionic acid,
3-biphenyl-4-yl-3-{2-[3-(3-((R)-1-naphthalen-1-ylethyl)ureido)propanoyl-amino]ethanoylamino}propionic acid,
3-biphenyl-4-yl-3-{2-[3-(3-((S)-1-naphthalen-1-ylethyl)ureido)propanoyl-amino]ethanoylamino}propionic acid,
3-biphenyl-4-yl-3-{2-[3-(3-naphthalen-1-ylureido)propanoylamino]-ethanoylamino}propionic acid,
3-biphenyl-4-yl-3-{2-[3-(3-naphthalen-2-ylureido)propanoylamino]-ethanoylamino}propionic acid,
3-biphenyl-4-yl-3-{2-[3-(3-benzylthioureido)propanoylamino]ethanoylamino}propionic acid,
3-biphenyl-4-yl-3-{2-[3-(3-(4-methylbenzyl)ureido)propanoylamino]-ethanoylamino}propionic acid,
3-biphenyl-4-yl-3-{2-[3-(3-(2,4-dichlorobenzyl)ureido)propanoylamino]-ethanoylamino}propionic acid,
3-biphenyl-4-yl-3-{2-[3-(3-(4-fluorobenzyl)ureido)propanoylamino]-ethanoylamino}propionic acid,
3-biphenyl-4-yl-3-{2-[3-(3-(3,4-dichlorobenzyl)ureido)propanoylamino]-ethanoylamino}propionic acid,
3-biphenyl-4-yl-3-{2-[3-(3-(2-chlorobenzyl)ureido)propanoylamino]-ethanoylamino}propionic acid,
3-biphenyl-4-yl-3-{2-[3-(3-(3-propyl)ureido)propanoylamino]ethanoylamino}propionic acid,
3-biphenyl-4-yl-3-{2-[3-(3-allylureido)propanoylamino]ethanoylamino}-propionic acid,
3-biphenyl-4-yl-3-[2-(5-ureidopentanoylamino)ethanoylamino]propionic acid,
3-biphenyl-4-yl-3-{2-[5-(3-ethylureido)pentanoylamino]ethanoylamino}propionic acid,
3-biphenyl-4-yl-3-{2-[5-(3-cyclohexylureido)pentanoylamino]ethanoyl-amino}propionic acid,
3-biphenyl-4-yl-3-{2-[5-(3-isopropylureido)pentanoylamino]ethanoyl-amino}propionic acid,
3-biphenyl-4-yl-3-{2-[5-(3-butylureido)pentanoylamino]ethanoylamino}propionic acid,
3-biphenyl-4-yl-3-{2-[5-(3-tert-butylureido)pentanoylamino]ethanoylamino}propionic acid,
3-biphenyl-4-yl-3-{2-[5-(3-methylthioureido)pentanoylamino]ethanoylamino}propionic acid,
3-biphenyl-4-yl-3-{2-[5-(3-phenylureido)pentanoylamino]ethanoylamino}propionic acid,
3-biphenyl-4-yl-3-{2-[5-(3-phenylethylureido)pentanoylamino]ethanoylamino}propionic acid,
3-biphenyl-4-yl-3-{2-[5-(3-(2-chlorophenyl)ureido)pentanoylamino]-ethanoylamino}propionic acid,
3-biphenyl-4-yl-3-{2-[5-(3-(3-chlorophenyl)ureido)pentanoylamino]-ethanoylamino}propionic acid,
3-biphenyl-4-yl-3-{2-[5-(3-(4-chlorophenyl)ureido)pentanoylamino]-ethanoylamino}propionic acid,
3-biphenyl-4-yl-3-{2-[5-(3-(2-methoxyphenyl)ureido)pentanoylamino]-30 ethanoylamino}propionic acid,
3-biphenyl-4-yl-3-{2-[5-(3-(4-methoxyphenyl)ureido)pentanoylamino]-ethanoylamino}propionic acid,
3-biphenyl-4-yl-3-{2-[5-(3-(3-methoxyphenyl)ureido)pentanoylamino]-ethanoylamino}propionic acid,
3-biphenyl-4-yl-3-{2-[4-(3-((R)-1-phenylethyl)ureido)butanoylamino]-ethanoylamino}propionic acid,
3-biphenyl-4-yl-3-{2-[4-(3-((S)-1-phenylethyl)ureido)butanoylamino]-ethanoylamino}propionic acid,
3-biphenyl-4-yl-3-{2-[4-(3-naphthalen-1-ylureido)butanoylamino]-ethanoylamino}propionic acid,
3-biphenyl-4-yl-3-{2-[4-(3-benzylthioureido)butanoylamino]ethanoylamino}propionic acid,
3-biphenyl-4-yl-3-{2-[4-(3-naphthalen-2-ylureido)butanoylamino]-ethanoylamino}propionic acid,
3-biphenyl-4-yl-3-{2-[4-(3-(4-methylbenzyl)ureido)butanoylamino]-ethanoylamino}propionic acid,
3-biphenyl-4-yl-3-{2-[4-(3-(3-methylbenzyl)ureido)butanoylamino]-ethanoylamino}propionic acid,
3-biphenyl-4-yl-3-{2-[4-(3-(2-methylbenzyl)ureido)butanoylamino]-ethanoylamino}propionic acid,
3-biphenyl-4-yl-3-{2-[4-(3-(2,4-dichlorobenzyl)ureido)butanoylamino]-ethanoylamino}propionic acid,
3-biphenyl-4-yl-3-{2-[4-(3-(4-fluorobenzyl)ureido)butanoylamino]-ethanoylamino}propionic acid,
3-biphenyl-4-yl-3-{2-[4-(3-(3,4-dichlorobenzyl)ureido)butanoylamino]-ethanoylamino}propionic acid,
3-biphenyl-4-yl-3-{2-[4-(3-(2-chlorobenzyl)ureido)butanoylamino]-ethanoylamino}propionic acid,
3-biphenyl-4-yl-3-{2-[4-(3-(4-methoxybenzyl)ureido)butanoylamino]ethanoylamino}propionic acid,
3-biphenyl-4-yl-3-{2-[4-(3-propylureido)butanoylamino]ethanoylamino}propionic acid,
3-biphenyl-4-yl-3-{2-[4-(3-allylureido)butanoylamino]ethanoylamino}-propionic acid,
3-biphenyl-4-yl-3-{2-[5-(3-((R)-1-phenylethyl)ureido)pentanoylamino]-ethanoylamino}propionic acid,
3-biphenyl-4-yl-3-{2-[5-(3-((S)-1-phenylethyl)ureido)pentanoylamino]-ethanoylamino}propionic acid,
3-biphenyl-4-yl-3-{2-[5-(3-naphthalen-1-ylureido)pentanoylamino]-ethanoylamino}propionic acid,
3-biphenyl-4-yl-3-{2-[5-(3-benzylthioureido)pentanoylamino]ethanoylamino}propionic acid,
3-biphenyl-4-yl-3-{2-[5-(3-naphthalen-2-ylureido)pentanoylamino]-ethanoylamino}propionic acid,
3-biphenyl-4-yl-3-{2-[5-(3-(4-methylbenzyl)ureido)pentanoylamino]-ethanoylamino}propionic acid,
3-biphenyl-4-yl-3-{2-[5-(3-(3-methylbenzyl)ureido)pentanoylamino]-ethanoylamino}propionic acid,
3-biphenyl-4-yl-3-{2-[5-(3-(2-methylbenzyl)ureido)pentanoylamino]-ethanoylamino}propionic acid,
3-biphenyl-4-yl-3-{2-[5-(3-(2,4-dichlorobenzyl)ureido)pentanoylamino]-ethanoylamino}propionic acid,
3-biphenyl-4-yl-3-{2-[5-(3-(4-fluorobenzyl)ureido)pentanoylamino]-ethanoylamino}propionic acid,
3-biphenyl-4-yl-3-{2-[5-(3-(3,4-dichlorobenzyl)ureido)pentanoylamino]-ethanoylamino}propionic acid,
3-biphenyl-4-yl-3{2-[5-(3-(2-chlorobenzyl)ureido)pentanoylamino]-ethanoylamino}propionic acid,
3-biphenyl-4-yl-3-{2-[5-(3-(4-methoxybenzyl)ureido)pentanoylamino]-30 ethanoylamino}propionic acid,
3-biphenyl-4-yl-3-{2-[5-(3-propylureido)pentanoylamino]ethanoylamino}propionic acid,
3-biphenyl-4-yl-3-{2-[5-(3-allylureido)pentanoylamino]ethanoylamino}-propionic acid,
3-biphenyl-4-yl-3-{2-[3-(9H-fluoren-9-yl-methoxycarbonylamino)-propanoylamino]ethanoylamino}propionic acid,
3-biphenyl-4-yl-3-{2-[4-(9H-fluoren-9-yl-methoxycarbonylamino)butanoyl-amino]ethanoylamino}propionic acid,
3-biphenyl-4-yl-3-{2-[5-(9H-fluoren-9-yl-methoxycarbonylamino)-pentanoylamino]ethanoylamino}propionic acid,
3-biphenyl-4-yl-3-[2-(3-ethoxycarbonylaminopropanoylamino)ethanoyl-amino]propionic acid
3-biphenyl-4-yl-3-[2-(3-benzyloxycarbonylaminopropanoylamino)-ethanoylamino]propionic acid,
3-biphenyl-4-yl-3-{2-[3-(2,2-dimethylpropoxycarbonylamino)propanoylamino]ethanoylamino}propionic acid,
3-biphenyl-4-yl-3-[2-(4-ethoxycarbonylaminobutanoylamino)ethanoylamino]propionic acid,
3-biphenyl-4-yl-3-[2-(4-benzyloxycarbonylaminobutanoylamino)ethanoylamino]propionic acid
3-biphenyl-4-yl-3-{2-[4-(2,2-dimethylpropoxycarbonylamino)butanoylamino]ethanoylamino}propionic acid,
3-biphenyl-4-yl-3-{2-[3-(9H-fluoren-9-yl-methoxycarbonylamino)-propanoylamino]ethanoylamino}propionic acid,
3-biphenyl-4-yl-3-{2-[4-(9H-fluoren-9-yl-methoxycarbonylamino)butanoylamino]ethanoylamino}propionic acid,
3-biphenyl-4-yl-3-{2-[5-(9H-fluoren-9-ylmethoxycarbonylamino)-pentanoylamino]ethanoylamino}propionic acid,
3-biphenyl-4-yl-3-[2-(3-ethoxycarbonylaminopropanoylamino)ethanoylamino]propionic acid,
3-biphenyl-4-yl-3-[2-(3-benzyloxycarbonylaminopropanoylamino)-ethanoylamino]propionic acid,
3-biphenyl-4-yl-3-{2-[3-(2,2-dimethylpropoxycarbonylamino)propanoylamino]ethanoylamino}propionic acid,
3-biphenyl-4-yl-3-[2-(4-ethoxycarbonylaminobutanoylamino)ethanoylamino]propionic acid,
3-biphenyl-4-yl-3-[2-(4-benzyloxycarbonylaminobutanoylamino)ethanoylamino]propionic acid or
3-biphenyl-4-yl-3-{2-[4-(2,2-dimethyl-propoxycarbonylamino)butanoylamino]ethanoylamino}propionic acid.

3. Process for the preparation of the compounds of the formula I according to claim 1, their stereoisomers and their salts and solvates; characterised in that
(a) a compound of the formula II 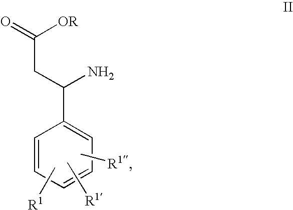
in which R is a protecting group, and R¹, R^{1′} and R^{1″} are as defined in formula I and in which, in the case where R¹, R^{1′} and/or R^{1″} have free hydroxyl or amino groups, these are in each case protected by a protecting group,
is reacted with a compound of the formula III 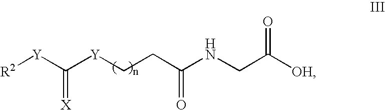
in which R² and n are as defined in formula I and in which, in the case where R² contains free hydroxyl and/or amino groups, these are in each case protected by protecting groups, and the protecting group R and any protecting groups present on R¹, R^{1′}, R^{1″} and/or R² are removed, or
(b) a compound of the formula IV 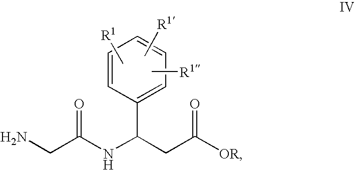
in which R is a protecting group, and R¹, R^{1′} and R^{1″} are as defined in formula I and in which, in the case where R¹, R^{1′} and/or R^{1″} contain free hydroxyl and/or amino groups, these are in each case protected by protecting groups,
is reacted with a compound of the formula V 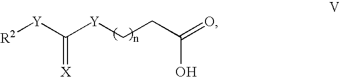
in which n and R² are as defined in formula I and in which, in the case where R² contains free hydroxyl and/or amino groups, these are in each case protected by protecting groups, and the protecting group R and any protecting groups present on R¹, R^{1′}, R^{1″} and/or R² are removed, or
(c) one or more radicals R¹, R^{1′}, R^{1″} and/or R² in a compound of the formula I are converted into one or more radicals R¹, R^{1′}, R^{1″} and/or R²
by, for example,
i) alkylating a hydroxyl group,
ii) hydrolysing an ester group to a carboxyl group,
iii) esterifying a carboxyl group,
iv) alkylating an amino group,
v) reacting an aryl bromide or iodide with boronic acids by a Suzuki coupling to give the corresponding coupling products, or
vi) acylating an amino group, or
(d) a compound of the formula VI 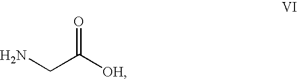
in which the free amino group is protected by protecting groups, is reacted with a compound of the formula II,
in which R is a protecting group and R¹, R^{1′} and R^{1″} are as defined in formula I and in which, in the case where R¹, R^{1′} and/or R^{1″} have free hydroxyl or amino groups, these are in each case protected by a protecting group,
to give a compound of the formula IV,
in which R is a protecting group and R¹, R^{1′} and R^{1″} are as defined in formula I and in which, in the case where R¹, R^{1″} and/or R^{1″} have free hydroxyl or amino groups, these are in each case protected by protecting groups,
the protecting group on the amino group is removed,
the compound of the formula IV is subsequently reacted as described in (b) with a compound of the formula V,
in which n and R² are as defined in formula I and in which, in the case where R² contains free hydroxyl and/or amino groups,
these are in each case protected by protecting groups,
and the protecting group R and any protecting groups present on R¹, R^{1′}, R^{1″} and/or R² are removed, and/or
a basic or acidic compound of the formula I is converted into one of its salts or solvates by treatment with an acid or base

4. Compounds of the formula I according to claim 1 or 2, their stereo-isomers and their physiologically acceptable salts or solvates as medicament active ingredients.

5. Compounds of the formula I according to claim 1 or 2, their stereoisomers and their physiologically acceptable salts or solvates as integrin agonists and/or antagonists.

6. Compounds of the formula I according to claim 1 or 2, their stereoisomers and their physiologically acceptable salts or solvates for use in combating illnesses.

7. Pharmaceutical preparation at least comprising a compound of the formula I according to claim 1 or 2, its stereoisomers and/or one of its physiologically acceptable salts or solvates.

8. Use of compounds of the formula I according to claim 1 or 2, their stereoisomers and/or their physiologically acceptable salts or solvates for the preparation of a medicament.

9. Use of compounds of the formula I according to claim 1 or 2, their stereoisomers and/or their physiologically acceptable salts or solvates for the prophylaxis and/or therapy of circulation disorders, pulmonary fibrosis, pulmonary embolism, thrombosis, in particular deep-vein thrombosis, cardiac infarction, arteriosclerosis, aneurysma dissecans, transient ischaemic attacks, apoplexia, angina pectoris, in particular unstable angina pectoris, pathological connecting tissue proliferation in organs or fibrosis, in particular pulmonary fibrosis, but also cystic fibrosis, dermatofibrosis, hepatic fibrosis, liver cirrhosis, urethrofibrosis, renal fibrosis, cardiac fibrosis, infantile endocardial fibrosis, pancreatic fibrosis, disturbed hornification of the skin, in particular leukoplakia, lichen planus and squamous cell carcinoma, tumour illnesses, such as tumour development, tumour angiogenesis or tumour metastasis, of solid tumours and those of the blood or immune system, for example tumours of the skin, squamous cell carcinoma, tumours of the blood vessels, of the gastro-intestinal tract, of the lung, of the breast, of the liver, of the kidney, of the spleen, of the pancreas, of the brain, of the testes, of the ovary, of the womb, of the vagina, of the muscles, of the bones, and those of the throat and head area, osteolytic illnesses, such as osteoporosis, hyperparathyroidism, Paget's disease, malign hypercalcaemia, incompatible blood transfusion, pathologically angiogenic disorders, such as, for example, inflammation, ophthalmological disorders, diabetic retinopathy, macular degeneration, myopia, corneal transplant, ocular histoplasmosis, rheumatic arthritis, osteoarthritis, rubeotic glaucoma, ulcerative colitis, Crohn's disease, atherosclerosis, psoriasis, restenosis, in particular after angioplasty, multiple sclerosis, pregnancy, absumptio placentaris, viral infection, bacterial infection, fungal infection, foot and mouth disease (FMD), HIV, anthrax, candida albicans, in the case of parasitic infestation, in the case of acute kidney failure and in the case of wound healing for supporting the healing process.
